(19) United States
(12) Patent Application Publication
(10) Pub. No.: US 2008/0161255 A1
(43) Pub. Date: Jul. 3, 2008

(54) Use of Parp Inhibitors for Prevention and Treatment of Diabetic and Insulin Resistance Complications

(76) Inventor: Michael Brownlee, New York, NY (US)

Correspondence Address: AMSTER, ROTHSTEIN & EBENSTEIN LLP, 90 PARK AVENUE, NEW YORK, NY, 10016, US

(21) Appl. No.: 10/558,532

(22) PCT Filed: May 27, 2004
(86) PCT No.: PCT/US04/16562

§ 371 Date: Feb. 12, 2007

Related U.S. Application Data
(60) Provisional application No. 60/474,520, filed on May 29, 2003.

(51) Int. Cl. A61K 48/00; A61K 38/17; A61K 39/395; A61K 31/455; A61K 31/454; A61K 31/51; A61K 31/165

(52) U.S. Cl. 514/44; 514/620; 514/300; 514/298; 514/400; 424/133.1; 530/387.1; 435/7.1; 514/276; 514/323; 514/356; 514/12

(57) ABSTRACT

The present invention provides methods of inhibiting the development or progression of atherosclerotic, microvascular, or neurologic disease due to diabetes or insulin resistance in a mammal, or conditions resulting therefrom. The methods involve specifically inhibitingpoly(ADP-ribose) polymerase (PARP) activity or accumulation in the mammal. Also provided are antibodies that specifically react with Nα-acetyl-Nδ (5-hydro-5-methyl)4-imidazolone. Additionally, the invention provides methods of monitoring the effectiveness of an anti-diabetic or anti-insulin resistance treatment or an anti-diabetic or anti-insulin resistance complication treatment in a mammal. The methods involve measuring ADP-ribosylated protein levels, or measuring methylglyoxyl AGE levels in the mammal using an antibodies that specifically react with Nα-acetyl-Nδ (5-hydro-5-methyl)4-imidazolone, or measuring GlcNAc-modified protein levels in the mammal.

### CROSS-REFERENCE TO RELATED APPLICATION

This application claims the benefit of U.S. Provisional Application No. 60/474,520, filed May 29, 2003.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided by the terms of Grant No. DK33861-17 awarded by the National Institutes of Health.

### BACKGROUND

(1) Field of the Invention

The present invention generally relates to treatment of diabetes and/or insulin resistance and methods of monitoring diabetes and/or insulin resistance treatments. More specifically, the invention provides novel methods of treating diabetes and/or insulin resistance to prevent the development or progression of the vascular and neurologic complications of diabetes and/or insulin resistance, and novel methods of monitoring those and other treatments of diabetes and/or insulin resistance.

(2) Description of the Related Art

### References Cited

Beckman, J. A., Goldfine, A. B., Gordon, M. B., Garrett, L. A. & Creager, M. A. (2002) Circ. Res. 90, 107-111.

Berg A H, Combs T P, Du X, Brownlee M, Scherer P E (2001) Nat. Med. 7, 947-953.

Bowman, K. J., Newell, D. R., Calvert, A. H., and Curtin, N. J. (2001) Br. J. Cancer 84, 106-112.

Brownlee, M. (2001) Nature 414, 813-820.

Brune, B. & Lapetina, E. G. (1995) Genet. Eng (N. Y.) 17, 149-164.

Brune, B., & Lapetina, E. G. (1996) Methods Enzymol. 269: 400-7.

Bucciarelli, L. G., Wendt, T., Qu, W., Lu, Y., Lalla, E., Rong, L. L., Goova, M. T., Moser, B., Kislinger, T., Lee, D. C. et al. (2002) Circulation 106, 2827-2835.

Burke, D. H., L. Scates, K. Andrews, and L. Gold (1996) J. Mol. Biol. 264, 650-666.

Connor, F. I. et al. (2001) Anal. Biochem. 293, 169-177.

Diabetes Control and Complications Trial Research Group (DCCTRG) (1993) N. Engl. J. Med. 329, 977-986.

Du, X. L., Edelstein, D., Rossetti, L., Fantus, I. G., Goldberg, H., Ziyadeh, F., Wu, J. & Brownlee, M. (2000) Proc. Natl. Acad. Sci. U. S. A 97, 12222-12226.

Du, X. L., Edelstein, D., Dimmeler, S., Ju, Q., Sui, C. & Brownlee, M. (2001).J. Clin. Invest 108, 1341-1348.

Du, X. L., Matsumura, T., Szabo, C., Edelstein, D. and Brownlee, M. (2002) American Diabetes Association 2002 Meeting, Abstract/Poster 707-P.

Eliasson, M. J., Sampei, K., Mandir, A. S., Hum, P. D., Traystman, R. J., Bao, J., Pieper, A., Wang, Z. Q., Dawson, T. M., Snyder, S. H. et al. (1997) Nat. Med. 3, 1089-1095.

Ellington, A. D., and J. W. Szostak (1990) Nature 346, 818-22.

Garcia, S. F., Virag, L., Jagtap, P., Szabo, E., Mabley, J. G., Liaudet, L., Marton, A., Hoyt, D. G., Murthy, K. G., Salzman, A. L. et al. (2001) Nat. Med. 7, 108-113.

Greb, A. and Bitsch, R. (1998) Int. J. Clin. Pharmacol. Ther. 36, 216-221.

Ha, H. C., Hester, L. D. & Snyder, S. H. (2002) Proc. Natl. Acad. Sci. U.S.A 99, 3270-3275.

Hagen T M, Ingersoll R T, Lykkesfeldt J, Liu J, Wehr C M, Vinarsky V, Bartholomew J C, Ames A B (1999) FASEB J. 13, 411-8

Hammes, H. P., Martin, S., Federlin, K., Geisen, K. & Brownlee, M. (1991) Proc. Natl. Acad. Sci. U.S.A. 88, 11555-11558.

Hammes, H. P., Du, X. L., Edelstein, D., Taguchi, T., Matsumura, T., Ju, J., Bierhaus, A., Nawroth, P., Hannak, D., Neumaier, M., Bergfeld, R., Giardino, I., and Brownlee, M. (2003) Nat. Med. 9, 294-299.

Hassa, P. O., Covic, M., Hasan, S., Imhof, R. & Hottiger, M. O. (2001) J. Biol. Chem. 276, 45588-45597.

Hirao, I., M. Spingola, D. Peabody, and A. D. Ellington (1998) Mol Divers. 4, 75-89.

Incara: see www.incara.com/catalytic.htm.

Inotek: see www.inotekcorp.com/news/Feb. 28, 2003.htm.

Ishii, H., Jirousek, M. R., Koya, D., Takagi, C., Xia, P., Clermont, A., Bursell, S. E., Kern, T. S., Ballas, L. M., Heath, W. F. et al. (1996) Science 272, 728-731.

Izumi, M., McDonald, M. C., Sharpe, M. A., Chatterjee, P. K., Thiemermann, C. (2002) Shock 18, 230-235.

Jaeger, J., T. Restle, and T. A. Steitz (1998) The EMBO Journal 17, 4535.

Kensch, O., B. A. Connolly, H. J. Steinhoff, A. McGregor, R. S. Goody, and T. Restle (2000) J Biol Chem. 275, 18271-8.

Kilhovd, B. K., Giardino, I., Torjesen, P. A., Birkeland, K. I., Berg, T J., Thornalley, P. J., Brownlee, M., and Hanssen, K. F. (2003) Metabolism 52, 163-167.

Koya, D., Haneda, M., Nakagawa, H., Isshiki, K., Sato, H., Maeda, S., Sugimoto, T., Yasuda, H., Kashiwagi, A., Ways, D. K. et al. (2000) FASEB J. 14,439-447.

Krynetski, E. Y., Krynetskaia, N. F., Bianchi, M. E., Evans, W. E. (2003) Cancer Res 63: 100-6.

Kurose, I., Saito, H., Miura, S., Ebinuma, H., Higuchi, H., Watanabe, N., Zeki, S., Nakamura, T., Takaishi, M. & Ishii, H. (1997) J. Clin. Invest 99, 867-878.

Lakka, H. et al. (2002) JAMA 288, 2709-2716.

Lee, A. Y., Chung, S. K. & Chung, S. S. (1995) Proc. Natl. Acad. Sci. U.S.A 92, 2780-2784.

Manna, S. K., Zhang, H. J., Yan, T., Oberley, L. W. & Aggarwal, B. B. (1998) J. Biol. Chem. 273, 13245-13254.

Mazzola, J. L.,& Sirover, M. A. (2002) Neurotoxicology. 23: 603-9.

McDonald, L. J. & Moss, J. (1993) Proc. Natl. Acad. Sci. U.S.A 90, 6238-6241.

Nakamura, S., Makita, Z., Ishikawa, S., Yasumura, K., Fujii, W., Yanagisawa, K., Kawata, T. & Koike, T. (1997) Diabetes 46, 895-899.

Nishikawa, T., Edelstein, D., Du, X. L., Yamagishi, S., Matsumura, T., Kaneda, Y., Yorek, M. A., Beebe, D., Oates, P. J., Hammes, H. P. et al. (2000) Nature 404, 787-790.

Pacher, P., Cziraki, A., Mabley, J. G., Liaudet, L., Papp, L. & Szabo, C. (2002a) Biochem. Pharmacol. 64, 1785-1791.

Pacher, P., Liaudet, L., Soriano, F. G., Mabley, J. G., Szabo, E. & Szabo, C. (2002b) Diabetes 51, 514-521.

Park, L., Raman, K. G., Lee, K. J., Lu, Y., Ferran, L. J., Jr., Chow, W. S., Stern, D. & Schmidt, A. M. (1998) Nat. Med. 4, 1025-1031.

Pieper, G. M. & Riaz, U. H. (1997) J. Cardiovasc. Pharmacol. 30, 528-532.

Pieper, A. A., Walles, T., Wei, G., Clements, E. E., Verma, A., Snyder, S. H. & Zweier, J. L. (2000) Mol. Med. 6, 271-282.

Rossetti, L. & Hu, M. (1993) J. Clin. Invest 92, 2963-2974.

Salvemini, D, Wang, Z. -Q., Zweier, J. L., Samouilov, A., Macarthur, H., Misko, T. P., Currie, M. G., Cuzzocrea, S., Sikorski, J. A, and Riley, D. P. (1999a) Science 286, 304-306.

Salvemini D., Riley D. P., Lennon, P. J., Wang, Z. Q., Currie, M. G., Macarthur, H., Misko, T. P. (1999b) Br. J. Pharmacol. 127, 685-692.

Sawa, A., Khan, A. A., Hester, L. D., & Snyder, S. H. (1997) Proc Natl Acad Sci USA 94: 11669-74.

Schmitz, H. D. (2001) Eur J Cell Biol 80, 419-27.

Schneider, D. J., J. Feigon, Z. Hostormsky, and L. Gold (1995) Biochemistry 34, 9599-9610.

Schraufstatter, I. U., Hinshaw, D. B., Hyslop, P. A., Spragg, R. G. & Cochrane, C. G. (1986) J. Clin. Invest 77, 1312-1320.

Shinohara, M., Thomalley, P. J., Giardino, I., Beisswenger, P., Thorpe, S. R., Onorato, J. & Brownlee, M. (1998) J. Clin. Invest 101, 1142-1147.

Sirover, M. A. (1997) J Cell Biochem. 66, 133-40.

Soriano, F. G., Virag, L., Jagtap, P., Szabo, E., Mabley, J. G., Liaudet, L., Marton, A., Hoyt, D. G., Murthy, K. G. K., Salzman, A. L., Southan, G. J., and Szabo, C. (2001) Nat. Med. 7, 108-113.

Soulis-Liparota, T., Cooper, M., Papazoglou, D., Clarke, B. & Jerums, G. (1991) Diabetes 40, 1328-1334.

Szabo, C. et al. (2002) Mol. Med. 8, 571-580.

Tao, Y., Howlett, A. & Klein, C. (1994) Eur. J. Biochem. 224, 447-454.

Thomas, K. W. (1986) J. Vet. Pharmacol. Ther. 9, 402-411.

Trevigen: sellers of PARP inhibitors. See www.trevigen.com.

Tuerk, C., and L. Gold (1992) Proc. Natl. Acad. Sci. USA 89: 6988-6992.

Tuerk, C., and L. Gold (1990) Science 249: 505-10.

Tunaru, S., Kero, J., Schaub, A., Wufka, C., Blaukat, A., Pfeffer, K., & Offermanns, S. (2003) Nat. Med. 9, 352-355.

UK Prospective Diabetes Study Group (UKPDSG) (1998) Lancet 352, 837-853.

Vedia, L., McDonald, B., Reep, B., Brune, B., Di Silvio, M., Billiar, T. R. & Lapetina, E. G. (1992) J. Biol. Chem. 267, 24929-24932.

Virag, L. & Szabo, C. (2002) Pharmacol. Rev. 54,375-429.

Soriano, F. G., Pacher, P., Mabley, J., Liaudet, L. & Szabo, C. (2001) Circ. Res. 89, 684-691.

Wakasaki, H., Koya, D., Schoen, F. J., Jirousek, M. R., Ways, D. K., Hoit, B. D., Walsh, R. A. & King, G. L. (1997) Proc. Natl. Acad. Sci. U.S.A 94, 9320-9325.

Wang-Fisher, Y. L., Han, J., & Guo, W. (2002) J. Endocrinol. 174, 267-272.

Wei, M., Gaskill, S. P., Haffner, S. M. & Stern, M. P. (1998) Diabetes Care 21, 1167-1172.

Welch, J. S., Ricote, M., Akiyama, T. E., Gonzalez, F. J. & Glass, C. K. (2003) Proc. Natl. Acad. Sci. U.S.A. online prepublication 10.1073/Proc. Natl. Acad. Sci. USA. 1031789100.

Zingarelli B, Day B J, Crapo J D, Salzman A L, Szabo C. (1997) Br. J. Pharmacol. 120, 259-267.

U.S. Pat. Re. 36,397.

U.S. Pat. No. 5,472,841.

U.S. Pat. No. 5,496,938.

U.S. Pat. No. 5,503,978.

U.S. Pat. No. 5,567,588.

U.S. Pat. No. 5,580,737.

U.S. Pat. No. 5,582,981.

U.S. Pat. No. 5,637,459.

U.S. Pat. No. 5,654,151.

U.S. Pat. No. 5,683,867.

U.S. Pat. No. 5,705,337.

U.S. Pat. No. 5,712,375.

U.S. Pat. No. 5,726,017.

U.S. Pat. No. 5,773,598.

U.S. Pat. No. 5,786,462.

U.S. Pat. No. 5,503,978.

U.S. Pat. No. 6,028,186.

U.S. Pat. No. 6,083,696.

U.S. Pat. No. 6,103,714.

U.S. Pat. No. 6,110,900.

U.S. Pat. No. 6,124,449.

U.S. Pat. No. 6,127,119.

U.S. Pat. No. 6,140,490.

U.S. Pat. No. 6,147,204.

U.S. Pat. No. 6,168,778.

U.S. Pat. No. 6,171,795.

U.S. Pat. No. 6,544,975 B1.

Diabetes causes a variety of pathological changes in capillaries, arteries, and peripheral nerves. Large prospective clinical studies in both type 1 and type 2 diabetic patients have shown that there is a strong relationship between the level of hyperglycemia and both onset and progression of diabetic microvascular complications in the retina, kidney, and peripheral nerve(DCCTRG, 1993; UKPDSG, 1998). Hyperglycemia also appears to have an important role in the pathogenesis of diabetic macrovascular disease (UKPDSG, 1998; Wei et al., 1998).

Although hyperglycemia is a risk factor for accelerated atherosclerosis, the recent Insulin Resistance and Atherosclerosis Study (IRAS) demonstrates that there is an even stronger link between insulin resistance itself and atherosclerosis, even in the absence of diabetes. This conclusion is supported by most studies of diabetes and atherosclerosis. The largest prospective trial examining glycemic control in type 2 diabetes, the UKPDS, did not find improved cardiovascular outcomes in the intensive control group, contrary to improvements seen in microvascular disease, suggesting that the intervention was not effective for macrovascular disease (UK Prospective Diabetes Study Group, 1998), and recently, cardiovascular disease was found to be increased in men with insulin resistance in the absence of baseline cardiovascular disease and diabetes (Lakka et al., 2002). Thus, in people with insulin resistance without diabetes, as well as in people with diabetes, metabolite-accelerated atherosclerosis is a major clinical problem. Two consequences of insulin resistance that are likely contributors are lipoprotein abnormalities and increased free fatty acid (FFA) flux.

Four major molecular mechanisms have been implicated in hyperglycemia-induced tissue damage: activation of protein kinase C (PKC) isoforms via de novo synthesis of the lipid second messenger diacylglycerol (DAG), increased hexosamine pathway flux, increased advanced glycation endproduct (AGE) formation, and increased polyol pathway flux. In aortic endothelial cells, hyperglycemia also activates the proinflammatory transcription factor NFκB. Recently, it has been shown that all of these mechanisms reflect a single hyperglycemia-induced process: overproduction of superoxide by the mitochondrial electron transport chain (Brownlee, 2001; Nishikawa et al., 2000). We have also recently discovered that increased free fatty acids activate the same pathways by the same mechanisms in aortic endothelial cells, thus providing an explanation for the pro-atherogenic effects of insulin resistance, as well as diabetes. However, the molecular mechanism by which this hyperglycemia (or free fatty acid)-induced overproduction of superoxide activates these different pathways of hyperglycemic (or free fatty acid-induced) damage has not been elucidated. There is thus a need for further elucidation of that mechanism and the utility of potential treatments directed toward countering that mechanism. The present invention satisfies that need.

### SUMMARY OF THE INVENTION

Accordingly, the inventor has succeeded in discovering that hyperglycemia-induced mitochondrial superoxide overproduction activates poly (ADP-ribose) polymerase (PARP). PARP activation, in turn, inhibits glyceraldehyde-3-phosphate dehydrogenase (GAPDH) activity which activates at least three of the major pathways of hyperglycemic damage in endothelial cells. Inhibiting PARP activity thus inhibits the mechanisms known to cause development of complications of diabetes and insulin resistance. See Examples 1 and 2. Compositions and methods related to these discoveries are also provided.

Thus, the present invention is directed to methods of inhibiting the development or progression of atherosclerotic, microvascular, or neurologic disease due to diabetes and/or insulin resistance in a mammal, or conditions resulting therefrom. The methods comprise specifically inhibiting poly(ADP-ribose) polymerase (PARP) activity or accumulation in the mammal for a time sufficient to inhibit the development or progression of the disease or condition.

In other embodiments, the invention is directed to antibody preparations comprising antibodies that specifically react with Nα-acetyl-Nδ(5-hydro-5-methyl)4-imidazolone.

Additionally, the invention is directed to methods of monitoring the effectiveness of an anti-diabetic or anti-insulin resistance treatment or an anti-diabetic or anti-insulin resistance complication treatment in a mammal. The methods comprise measuring ADP-ribosylated protein levels in the mammal before and after the treatment, where ADP-ribosylated protein levels after the treatment lower than ADP-ribosylated protein levels before the treatment indicates that the treatment is effective.

In related embodiments, the invention is also directed to other methods of monitoring the effectiveness of an anti-diabetic or anti-insulin resistance treatment or anti-diabetic or anti-insulin resistance complication treatment in a mammal. These methods comprise measuring methylglyoxyl AGE levels in the mammal using an antibody that specifically reacts with Nα-acetyl-Nδ(5-hydro-5-methyl)4-imidazolone, where methylglyoxyl AGE levels after the treatment lower than methylglyoxyl AGE levels before the treatment indicates that the treatment is effective.

In other related embodiments, the invention is directed to additional methods of monitoring the effectiveness of an anti-diabetic or anti-insulin resistance treatment or anti-diabetic or anti-insulin resistance complication treatment in a mammal. In these embodiments, the methods comprise measuring GlcNAc-modified protein levels in the mammal, where GlcNAc-modified protein levels after the treatment lower than GlcNAc-modified protein levels before the treatment indicates that the treatment is effective.

### BRIEF DESCRIPTION OF THE DRAWINGS

FIG. 1 is a graph of experimental data establishing that GAPDH antisense oligonucleotides reduce GAPDH activity in bovine aortic endothelial cells. Each bar represents the mean ±SEM of 4 separate experiments. Asterisk, P<0.01 compared to cells incubated in 5 mM glucose alone.

FIG. 2 is four graphs of experimental data showing the effect of GAPDH antisense oligonucleotides on pathways of hyperglycemic damage in bovine aortic endothelial cells. Panel a. shows PKC activation caused by GAPDH antisense; Panel b. shows hexosamine pathway activation caused by GAPDH antisense; Panel c. shows intracellular AGE formation caused by GAPDH antisense; Panel d. shows NFκB activation caused by GAPDH antisense. Asterisk, P<0.01 compared to cells incubated in 5 mM glucose. For a.-c., each bar represents the mean ±SEM of 4 separate experiments. For d., each bar represents the mean ±SEM of fluorescence from 40 cells measured in an in situ DNA-protein binding assay.

FIG. 3 is two graphs of experimental data showing the effect of genes that alter mitochondrial superoxide production and of PARP inhibition on poly(ADP-ribosyl)ation of GAPDH (Panel a.), and GAPDH activity (Panel b.), in bovine aortic endothelial cells. Cells were incubated in 5 mM glucose or 30 mM glucose alone, in 30 mM glucose plus either control, UCP-1- or MnSOD-expressing adenoviral vectors, and in 30 mM glucose plus 3 mM PJ34. Each bar represents the mean ±SEM of 4 separate experiments. Asterisk, P<0.01 compared to cells incubated in 5 mM glucose alone. Only the 30 mM glucose, and the 30 mM glucose+vector treatments were different at P<0.01 from the 5 mM glucose alone treatment.

FIG. 4 is a graph of experimental data showing the effect of hyperglycemia and genes that alter mitochondrial superoxide production on PARP activity in bovine aortic endothelial cells. Cells were incubated in 5 mM glucose or 30 mM glucose alone, and in 30 mM glucose plus either control, UCP-1- or MnSOD-expressing adenoviral vectors. Each bar represents the mean ±SEM of 4 separate experiments. Asterisk, P<0.01 compared to cells incubated in 5 mM glucose alone. Only the 30 mM glucose, and the 30 mM glucose+vector treatments were different at P<0.01 from the 5 mM glucose treatment.

FIG. 5 is a set of micrographs and a graph of experimental data showing the effect of hyperglycemia and genes that alter mitochondrial superoxide production on DNA strand breaks in bovine aortic endothelial cells. Cells were incubated in 5 mM glucose or 30 mM glucose alone, or in 30 mM glucose plus either control, UCP-1- or MnSOD-expressing adenoviral vectors. Panel a., fluorescent micrographs from single cell electrophoresis assay. Panel b., Quantitation of DNA strand breaks from single cell electrophoresis assay. Each bar represents the mean ±SEM of 40 cells for each incubation condition. The bars in Panel b., from left to right, correspond to micrographs a-e, respectively, of Panel a. Asterisk, P<0.01 compared to cells incubated in 5 mM glucose alone. Only the 30 mM glucose, and the 30 mM glucose+vector treatments were different at P<0.01 from the 5 mM glucose treatment.

FIG. 6 is four graphs of experimental data showing the effect of PARP inhibition on hyperglycemia-induced pathways of vascular damage in bovine aortic endothelial cells. Panel a, PKC activation; Panel b, hexosamine pathway activation; Panel c, intracellular AGE formation; Panel d, NκB activation. Asterisk, P<0.01 compared to cells incubated in 5 mM glucose. For Panels a-c, each bar represents the mean ±SEM of 4 separate experiments. For Panel d, each bar represents the mean ±SEM of fluorescence from 40 cells measured in an in situ DNA-protein binding assay. In each graph, only the 30 mM glucose, and the 30 mM glucose+vector treatments were different at P<0.01 from the 5 mM glucose treatment.

FIG. 7 is a graph of experimental data showing the effects of various concentrations of oleic acid on superoxide generation in bovine aortic endothelial cells.

FIG. 8 is a graph of experimental data showing the effects of inhibitors of free fatty acid (FFA) oxidation (TDGA), UCP-1, and MnSOD on FFA-induced superoxide generation in bovine aortic endothelial cells.

FIG. 9 is a graph of experimental data showing the effect of nicotinic acid (a free fatty acid release inhibitor) and TBAP (a superoxide dismutase mimetic) treatment on reactive oxygen species (ROS)-induced inactivation of arterial prostacyclin synthase activity in non-diabetic, insulin resistant Fatty Zucker Rats.

FIG. 10 is graphs of experimental data showing the correlation of glyceraldehyde-3-phosphate dehydrogenase (GAPDH) activity with the degree of GAPDH poly(ADP-ribosyl)ation in lymphocytes from normal human volunteers during and after a 6-hour hyperglycemic clamp.

FIG. 11 is a graph of experimental data showing the activation of the enzyme poly(ADPribose)polymerase by free fatty acids (oleic acid).

### DETAILED DESCRIPTION OF THE INVENTION

The present invention is based in part on the discovery that hyperglycemia-induced mitochondrial superoxide overproduction activates poly(ADP-ribose) polymerase (PARP). PARP activation, in turn, inhibits glyceraldehyde-3-phosphate dehydrogenase (GAPDH) activity, which activates at least three of the major pathways of hyperglycemic damage in endothelial cells. Inhibiting PARP activity thus inhibits the development of complications of diabetes. See Example 1, showing PARP inhibition inhibits the activation of protein kinase C isoforms, hexosamine pathway flux, advanced glycation endproduct formation, and NFκB activation by inhibiting GAPDH activity. Because insulin resistance also causes many similar pathologic endothelial changes by similar mechanisms (See, e.g., Example 2), PARP inhibition also inhibits the development of complications of insulin resistance.

Accordingly, in some embodiments, the invention is directed to methods of inhibiting the development of atherosclerotic, microvascular, or neurologic disease due to diabetes or insulin resistance in a mammal, or conditions resulting therefrom. The methods comprise specifically inhibiting poly(ADP-ribose) polymerase (PARP) activity or accumulation in the mammal for a time sufficient to inhibit the development of the disease or condition. As used herein, “or” in phrases such as “diabetes or insulin resistance” or “atherosclerotic, microvascular, or neurologic disease” includes the cases where both conditions occur, e.g., where diabetes and insulin resistance are involved.

In these embodiments, the disease or condition includes any atherosclerotic, microvascular or neurologic disease caused by diabetes or insulin resistance or conditions resulting therefrom, now known or later discovered, because PARP inhibition would be expected to be effective against any such disease or condition. In preferred embodiments, the disease or condition is coronary disease, myocardial infarction, atherosclerotic peripheral vascular disease, cerebrovascular disease, stroke, retinopathy, renal disease, neuropathy, and/or cardiomyopathy, since those are currently the most important diseases or conditions resulting from diabetes or insulin resistance. In the most preferred embodiments, the condition is retinopathy (resulting from diabetes).

These embodiments are useful for treatment of any mammal. In preferred embodiments, the mammal is a human or a mouse.

These methods are not narrowly limited to any particular means of inhibiting PARP activity, since any means of inhibiting PARP would be expected to be useful. In preferred embodiments, PARP is inhibited by administering, to the mammal, compositions comprising a PARP inhibitor, a nucleic acid or mimetic that specifically inhibits transcription or translation of the PARP gene, or a compound that specifically binds to the PARP.

The above-described compositions can be formulated without undue experimentation for administration to a mammal, including humans, as appropriate for the particular application. Additionally, proper dosages of the compositions can be determined without undue experimentation using standard dose-response protocols.

Accordingly, the compositions designed for oral, lingual, sublingual, buccal and intrabuccal administration can be made without undue experimentation by means well known in the art, for example with an inert diluent or with an edible carrier. The compositions may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

The compositions of the present invention can easily be administered parenterally such as for example, by intravenous, intramuscular, intrathecal or subcutaneous injection. Parenteral administration can be accomplished by incorporating the compositions of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the composition in the glycerin, mixing the heated glycerin after which purified water may be added, and pouring the hot mixture into a suppository mold.

Transdermal administration includes percutaneous absorption of the composition through the skin. Transdermal formulations include patches (such as the well-known nicotine patch), ointments, creams, gels, salves and the like.

The present invention includes nasally administering to the mammal a therapeutically effective amount of the composition. As used herein, nasally administering or nasal administration includes administering the composition to the mucous membranes of the nasal passage or nasal cavity of the patient. As used herein, pharmaceutical compositions for nasal administration of a composition include therapeutically effective amounts of the composition prepared by well-known methods to be administered, for example, as a nasal spray, nasal drop, suspension, gel, ointment, cream or powder. Administration of the composition may also take place using a nasal tampon or nasal sponge.

Thus, in some preferred embodiments, PARP activity is inhibited by administering to the mammal a PARP inhibitor. As used herein, a PARP inhibitor is a small molecular weight (less than, about 1000 daltons) compound that specifically inhibits PARP. Examples include PJ34 (Soriano et al., 2001; Pacher et al., 2002b), 3-aminobenzamide (Trevigen), 4-amino-1,8-naphthalimide (Trevigen), 6(5H)-phenanthridinone (Trevigen), benzamide (U.S. Pat. Re. 36,397), INO-1001 (Inotek), and NU1025 (Bowman et al.). In preferred embodiments, the PARP inhibitor is PJ34, INO-1001, or 3-aminobenzamide.

In other embodiments, PARP activity is inhibited by administering to the mammal a nucleic acid or mimetic that specifically inhibits transcription or translation of the PARP gene. In preferred embodiments, these nucleic acids or mimetics are an antisense complementary to mRNA of the PARP gene, a ribozyme capable of specifically cleaving the mRNA of the PARP gene, or an RNAi molecule complementary to a portion of the PARP gene. Preferably, the mRNA of the PARP gene is at least 80% homologous to the human PARP mRNA as provided in SEQ ID NO: 1 (from GenBank NM 001618). More preferably, the mRNA of the PARP gene is at least 90%, even more preferably 95%, and most preferably at least 99%, or completely complementary to SEQ ID NO: 1. In these embodiments, the antisense molecule, ribozyme, or RNAi molecules an be comprised of nucleic acid (e.g., DNA or RNA) or nucleic acid mimetics (e.g., phosphorothioate mimetics) as are known in the art. These embodiments are not narrowly limited to any particular means of administration of the nucleic acids or mimetics. Such administration can include, for example, administration of the nucleic acid directly (e.g., intravenously) or by administration of a vector that expresses the nucleic acid. Many such vectors are well known, and include naked DNA vectors and viral vectors (e.g., lentiviral or adenoviral vectors). The skilled artisan can determine the most appropriate method of administration for any particular compound without undue experimentation.

Thus, the nucleic acid or mimetic in some of these embodiments is an antisense molecule complementary to a portion of a mammalian PARP gene. Since antisense technology is well developed, an effective antisense for any mammalian PARP gene can be made without undue experimentation.

The nucleic acid or mimetic can also be a ribozyme capable of specifically cleaving the mRNA of the PARP gene. Since ribozyme technology is well developed, an effective ribozyme for any mammalian PARP gene can also be made without undue experimentation.

Additionally, the nucleic acid or mimetic is an RNAi molecule. As is well known, an RNAi (including siRNA) molecule is a short double stranded nucleic acid that interferes with transcription or translation of a homologous gene. Since RNAi technology is well developed, an effective RNAi molecule for any mammalian PARP gene can also be made without undue experimentation.

In further embodiments, the PARP activity is inhibited by administration of a compound that specifically binds to the PARP. In these embodiments, the compound capable of binding the PARP can be any compound capable of interfering with PARP activity, e.g., by preventing the PARP substrate from interacting with the PARP. In preferred embodiments, the compound is an antibody or an aptamer.

Methods of making antibodies that inhibit enzyme activity are routine, and the skilled artisan would expect that such an antibody could be made to any PARP without undue experimentation. The antibodies can be from a polyclonal, monoclonal, or recombinant source. As used herein, “antibodies” also include fragments of whole antibodies that comprises a typical immunoglobulin antigen binding site (e.g., Fab or Fab2). The antibodies can also be of any vertebrate (e.g., mouse, chicken, rabbit, goat or human), or of a mixture of vertebrates (e.g., humanized mouse).

Aptamers are single stranded oligonucleotides or oligonucleotide analogs that bind to a particular target molecule, such as a protein or a small molecule (e.g., a steroid or a drug, etc.). Thus, aptamers are the oligonucleotide analogy to antibodies. However, aptamers are smaller than antibodies, generally in the range of 50-100 nt. Their binding is highly dependent on the secondary structure formed by the aptamer oligonucleotide. Both RNA and single stranded DNA (or analog), aptamers are known. See, e.g., Burke et al., 1996; Ellington and Szostak 1990; Hirao et al., 1998; Jaeger et al., 1998; Kensch et al, 2000; Schneider et al., 1995; and U.S. Pat. Nos. 5,773,598; 5,496,938; 5,580,737; 5,654,151; 5,726,017; 5,786,462; 5,503,978; 6,028,186; 6,110,900; 6,124,449; 6,127,119; 6,140,490; 6,147,204; 6,168,778; and 6,171,795.

Aptamers that bind to virtually any particular target can be selected by using an iterative process called SELEX, which stands for Systematic Evolution of Ligands by EXponential enrichment (Burke et al., 1996; Ellington and Szostak, 1990; Schneider et al., 1995; Tuerk and Gold, 1992; Tuerk and Gold, 1990). Several variations of SELEX have been developed which improve the process and allow its use under particular circumstances. See, e.g., U.S. Pat. Nos. 5,472,841; 5,503,978; 5,567,588; 5,582,981; 5,637,459; 5,683,867; 5,705,337; 5,712,375; and 6,083,696. Thus, the production of aptamers that binds to any particular protein, including PARP, requires no undue experimentation.

The methods described above can be employed together with other methods of treating diabetes or insulin resistance or their complications, e.g., insulin treatment, or another treatment directed to inhibiting the development of atherosclerotic, microvascular, or neurologic disease due to diabetes, or conditions resulting therefrom. Nonlimiting and preferred examples of such treatments include activating transketolase, reducing superoxide, and inhibiting excessive release of free fatty acids in the mammal.

Thus, in some embodiments, the methods of inhibiting PARP described above is combined with a method comprising the activation of transketolase. In preferred embodiments, the transketolase is activated by administering a lipid-soluble thiamine derivative to the mammal. Nonlimiting and preferred examples of lipid-soluble thiamine derivatives useful in these methods are benfotiamine (Hammes et al., 2003; Greb & Bitsch, 1998), thiamine propyl disulfide (Thomas, 1986), and thiamine tetrahydrofurfuryl disulfide (Greb & Bitsch, 1998).

In other embodiments, the methods of inhibiting PARP described above is combined with a method comprising reducing superoxide or peroxynitrite in the mammal. As is known, peroxynitrite forms from superoxide and nitric oxide, and likely contributes or is responsible for superoxide-induced DNA damage (Szabo et al., 2002). The superoxide can be reduced in the mammal by any means known in the art, e.g., administration of an antioxidant. In preferred embodiments, the superoxide is reduced by administering, to the mammal, R-alpha-lipoic acid (Hagen et al., 1999), FP15 (a peroxynitrite decomposition catalyst—see, e.g., Szabo et al., 2002), a superoxide dismutase mimetic or a catalase mimetic. Several superoxide dismutase mimetics an d catalase mimetics are known in the art, e.g., MnTBAP (U.S. Pat. No. 6,103,714), ZnTBAP (Zingarelli et al., 1997), SC-55858 (Salvemini et al., 1999b), EUK-134 (Izumi et al., 2002), AEOL 10112 (Incara), AEOL 10113 (Incara), AEOL 10150 (Incara; U.S. Pat. No. 6,544,975 B1) and M40403 (Salvemini et al., 1999a). Preferably, the superoxide dismutase mimetic or catalase mimetic is M40403, MnTBAP, AEOL 10112, AEOL 10113, AEOL 10150, or ZnTBAP.

In additional embodiments, the methods of inhibiting PARP described above is combined with a method comprising inhibiting excessive release of free fatty acids in the mammal. Preferably, excessive release of free fatty acids is inhibited by administering, to the mammal, a thiazolidinedione (Welch et al., 2003), nicotinic acid (Tunaru et al., 2003), adiponectin (Berg et al., 2001) and acipimox (Wang-Fisher, 2002).

The inventor has also succeeded in developing methods for producing antibodies that specifically react with Nα-acetyl-Nδ(5-hydro-5-methyl)-4-imidazolone (Kilhovd et al., 2003). These antibodies are useful for measuring methylglyoxyl AGE levels in the mammal (Id.) Preferably, the antibodies are sufficiently specific for Nα-acetyl-Nδ(5-hydro-5-methyl)-4-imidazolone that they do not react appreciably with Nα-acetyl-argpyrimidine, bis(Nα-acetyl)lys-4-methyl-imidazolium chloride or carboxyethyllysine.

The antibodies can be from a polyclonal, monoclonal, or recombinant source. As used herein, “antibodies” also include a fragment of a whole antibody that comprises a typical immunoglobulin antigen binding site (e.g., Fab or Fab2). The antibodies can also be of any vertebrate (e.g., mouse, chicken, rabbit, goat or human), or of a mixture of vertebrates (e.g., humanized mouse). In preferred embodiments, the antibodies are monoclonal antibodies, for example the IG7 antibodies described in Kilhovd et al. (2003).

The inventor has also succeeded in discovering that the effectiveness of an anti-diabetic or anti-insulin resistance treatment or an anti-diabetic or anti-insulin resistance complication treatment in a mammal (as described above) can be monitored by measuring ADP-ribosylated protein levels in the mammal before and after the treatment, wherein ADP-ribosylated protein levels after the treatment lower than ADP-ribosylated protein levels before the treatment indicates that the treatment is effective. The effectiveness of this treatment is based on the above-described realization that PARP activation is a major mechanism by which excess superoxide leads to diabetic complications. Since PARP poly(ADP-ribosyl)ates proteins, the amount of ADP-ribosylated protein is directly reflective of PARP activation.

Thus, the invention is also directed to methods of monitoring the effectiveness of an anti-diabetic or anti-insulin resistance treatment or an anti-diabetic or anti-insulin resistance complication treatment in a mammal (including humans). The methods comprise measuring ADP-ribosylated protein levels in the mammal before and after the treatment. In these methods, ADP-ribosylated protein levels after the treatment that are lower than ADP-ribosylated protein levels before the treatment indicates that the treatment is effective.

In these methods, ADP-ribosylated protein levels can be determined by any means known in the art, for example by immunoassay using anti-poly(ADPribose) antibodies. Immunoassay technology is well developed, and the skilled artisan could develop any appropriate immunoassay for ADP-ribosylation without undue experimentation. The methods can comprise measuring ADP-ribosylation of all proteins, e.g., in a serum or tissue sample. However, in preferred embodiments, ADP-ribosylation is measured in only one protein, e.g., by immunoprecipitation followed by western blotting. Preferably, the protein is GAPDH, since ADP-ribosylation of GAPDH is directly involved in the vascular and neurologic pathology of diabetes, as previously discussed.

These methods are not narrowly limited to the monitoring of any particular anti-diabetic or anti-insulin resistance treatment or anti-diabetic or anti-insulin resistance complication treatment. Any of the above described treatments, or any other treatment for diabetes or insulin resistance, now known or later discovered, can be usefully monitored by these methods. Nonlimiting examples include insulin administration, and any of the above-described treatments including inhibiting PARP activity, administration of a compound that activates transketolase, administration of a compound that reduces superoxide, and administration of a compound that inhibits excessive release of free fatty acids.

The inventor has also succeeded in discovering that the effectiveness of an anti-diabetic or anti-insulin resistance treatment or an anti-diabetic or anti-insulin resistance complication treatment in a mammal (as described above) can be monitored by measuring methylglyoxyl AGE levels in the mammal using an antibodies that specifically react with Nα-acetyl-Nδ(5-hydro-5-methyl)-4-imidazolone. Since methylglyoxyl AGE levels directly reflect the severity of diabetes and its complications, the determination that methylglyoxyl AGE levels have gone down indicates that the treatment is effective.

Thus, in these embodiments, the invention is directed to methods of monitoring the effectiveness of an anti-diabetic or anti-insulin resistance treatment or an anti-diabetic or anti-insulin resistance complication treatment in a mammal (including humans). The methods comprise measuring methylglyoxyl AGE levels in the mammal using antibodies that specifically react with Nα-acetyl-Nδ(5-hydro-5-methyl)-4-imidazolone. In these methods, methylglyoxyl AGE levels after the treatment lower than methylglyoxyl AGE levels before the treatment indicates that the treatment is effective.

In these methods, ADP-ribosylated protein levels can be determined by any means known in the art using the antibodies, for example by immunoassay. Any sample from the mammal that comprises proteins can be used for this assay. Preferably, the sample is a serum sample. The antibodies that specifically react with Nα-acetyl-Nδ(5-hydro-5-methyl)-4-imidazolone are described more fully above. Preferably, the antibodies are the IG7 monoclonal antibodies described above.

These methods are also not narrowly limited to the monitoring of any particular anti-diabetic or anti-insulin resistance treatment or anti-diabetic or anti-insulin resistance complication treatment. Any of the above described treatments, or any other treatment for diabetes or insulin resistance, now known or later discovered, can be usefully monitored by these methods. Nonlimiting examples include insulin administration, and any of the above described treatments including inhibiting PARP activity, administration of a compound that activates transketolase, administration of a compound that reduces superoxide, and administration of a compound that inhibits excessive release of free fatty acids.

The inventor has also succeeded in discovering that the effectiveness of an anti-diabetic or anti-insulin resistance treatment or an anti-diabetic or anti-insulin resistance complication treatment in a mammal (as described above) can be monitored by measuring GlcNAc-modified protein levels in the mammal. Since GlcNAc-modified protein levels directly reflect the severity of glucose or free fatty acid-induced complication-causing PARP activation, the determination that levels GlcNAc-modified protein levels have gone down indicates that the treatment is effective.

Therefore, in these embodiments, the invention is directed to methods of monitoring the effectiveness of an anti-diabetic or anti-insulin resistance treatment or an anti-diabetic or anti-insulin resistance complication treatment in a mammal (including humans). The methods comprise measuring GlcNAc-modified protein levels in the mammal. In these methods, GlcNAc-modified protein levels after the treatment that are lower than GlcNAc-modified protein levels before the treatment indicates that the treatment is effective.

In these methods, GlcNAc-modified protein levels can be determined by any means known in the art. Preferably, GlcNAc-modified protein levels are determined using antibodies specific for GlcNAc-modified proteins. See, e.g., Conner et al., 2001, describing monoclonal antibody CTD110.6.

Any sample from the mammal that comprises proteins can be used for this assay. Preferably, the sample is a serum sample.

These methods are also not narrowly limited to the monitoring of any particular anti-diabetic or anti-insulin resistance treatment or anti-diabetic or anti-insulin resistance complication treatment. Any of the above described treatments, or any other treatment for diabetes or insulin resistance, now known or later discovered, can be usefully monitored by these methods. Nonlimiting examples include insulin administration, and any of the above described treatments including inhibiting PARP activity, administration of a compound that activates transketolase, administration of a compound that reduces superoxide, and administration of a compound that inhibits excessive release of free fatty acids.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

### EXAMPLE 1

### Inhibition of GAPDH Activity by Poly(ADP-ribose) Polymerase Activates Three Major Pathways of Hyperglycemic Damage in Endothelial Cells

### Example Summary

In this report, we show that hyperglycemia-induced overproduction of superoxide by the mitochondrial electron transport chain activates the three major pathways of hyperglycemic damage found in aortic endothelial cells (activation of protein kinase C isoforms, hexosamine pathway flux, and advanced glycation endproduct formation) by inhibiting GAPDH activity. In bovine aortic endothelial cells, GAPDH antisense oligonucleotides activated each of the pathways of hyperglycemic vascular damage in cells cultured in 5 mM glucose to the same extent as that induced by culturing cells in 30 mM glucose. Hyperglycemia-induced GAPDH inhibition was found to be a consequence of poly(ADP-ribosyl)ation of GAPDH by poly(ADP-ribose) polymerase (PARP), which was activated by DNA strand breaks produced by mitochondrial superoxide overproduction. Both the hyperglycemia-induced decrease in activity of GAPDH and its poly(ADP-ribosyl)ation were prevented by overexpression of either uncoupling protein-1 (UCP-1) or manganese superoxide dismutase (MnSOD), which decrease hyperglycemia-induced superoxide. Overexpression of UCP-1 or MnSOD also prevented hyperglycemia-induced DNA strand breaks and activation of PARP. Hyperglycemia-induced activation of each of the pathways of vascular damage was completely inhibited by blocking PARP activity with the PARP inhibitor PJ34. The demonstration that inhibition of PARP completely blocks hyperglycemia-induced activation of multiple pathways of vascular damage suggests that PARP inhibitors might have unique clinical efficacy in preventing the development and progression of diabetic complications.

Portions of these results were presented at the American Diabetes Association Annual Meeting, Jun. 14-18, 2002, poster 707-P (Du et al., 2002).

### Introduction

Since hyperglycemia-induced overproduction of superoxide significantly inhibits GAPDH activity (Du et al., 2000), we hypothesized that this inhibition would activate all the pathways of hyperglycemic damage by diverting upstream glycolytic metabolites into these signaling pathways. To test this hypothesis, we first evaluated the effect of inhibition of GAPDH activity by antisense ODN on the activity of each of these pathways in aortic endothelial cells cultured in 5 mM glucose. Since aldose reductase activity is extremely low in aortic endothelial cells, this pathway was not investigated.

We next investigated the mechanism by which hyperglycemia-induced overproduction of superoxide inhibits GAPDH activity in vivo. Although GAPDH activity can be inhibited by a number of covalent modifications in in vitro systems, including direct oxidative modification of protein thiols, nitric oxide dependent binding of NAD+, and monoADP-ribosylation (Vedia et al., 1992; McDonald & Moss, 1993; Brune & Lapetina, 1995), the physiologic importance of each of these remains unclear. Since hyperglycemia-induced loss of endothelium-dependent vasodilatation can be normalized by inhibition of poly(ADP-ribose)polymerase (Garcia et al., 2001), we examined first the relationship between hyperglycemia-induced reactive oxygen formation, poly(ADP-ribosyl)ation, and GAPDH activity. Since poly (ADP-ribose) polymerase (PARP) is activated by single or double strand breaks in DNA, we also examined the relationship between hyperglycemia-induced reactive oxygen formation and DNA strand breaks and consequent activation of PARP.

Finally, we sought to determine whether this sequence of events explained the activation of pathways of hyperglycemic damage in aortic endothelial cells by examining the effect of PARP inhibition on hyperglycemia-induced activation of all these pathways in aortic endothelial cells.

### Materials and Methods

Materials. Eagle's MEM, nonessential amino acids, and antibiotics were from Gibco (Grand Island, N.Y.). FBS was from Hyclone (Logen, Utah). Manganese (III) tetrakis(4-benzoic acid) porphyrin (Mn-TBAP) was from Calbiochem (La Jolla Calif.). Fluorescent oligos used in the NFκB assay were obtained from Operon Technologies Inc (Alameda, Calif.). Monoclonal anti-poly(ADPribose) IgG (10H) was from Alexis (Carlsbad Calif.). Protein A Sepharose was from Amersham Pharmacia.

Cell culture conditions. Confluent bovine aortic endothelial cells (passage 4-10) were maintained in Eagle's MEM containing 0.4% FBS, essential and non-essential amino acid and antibiotics. Cells were incubated for 3 days prior to determination of PKC activity, 48 hr prior to determination of hexosamine pathway activity, for 5 days prior to determination of advanced glycation end product formation, and for 6 hr prior to determination of NFκB activation. Cells were infected with adenoviruses 48 hr prior to addition of 30 mM glucose.

Oligonucleotide synthesis and treatment of cells. Phosphorothioate oligonucleotides were synthesized by Operon Technologies Inc.(Alameda, Calif.). The S-antisense GAPDH had the following sequence: 5′-G*TAGAAGCAGGGATGATAT*T-3′. Scrambled oligonucleotides (5′-G*AATAAGTGATACGGATGT*G-3′) were used as controls. Oligo solutions were prepared in 10 mM TRIS buffer, pH 7.4, containing 1 mM EDTA NaCl. 36.3 ml of oligonucleotide was mixed with 16.3 ml of polyethylenimine solution and 945 ml of media, and the solution was added to the cells for 2 hr.

GAPDH activity. BAECs were grown to confluency, harvested by using trypsin-EDTA after washing twice with PBS, and resuspended in lysis buffer. The cytosolic fraction was prepared by centrifuging the lysate at 100,000×g at 4° C. for 30 min. Protein was measured by using the Bio-Rad Coomassie Plus Protein Assay System. GAPDH activity was determined as described previously (Garcia et al., 2001; Tau et al., 1994).

Adenoviral vectors. Rat UCP-1 sense and antisense cDNAs were provided by D. Ricquier, Centre National de la Recherche Scientifique-Unite Propre 1511, Meudon, France, and human MnSOD cDNA was provided by L. Oberley, University of Iowa College of Medicine. The cDNAs were cloned into the shuttle vector pAd5CMVK-NpA and adenoviral vectors were prepared by the Gene Transfer Vector Core at the University of Iowa. Cells were infected at an MOI of 500 for 2 hr.

Ouantitation of DNA strand breaks. DNA strand breaks were detected with a single-cell gel electrophoresis assay (CometAssay, TREVIGEN, Gaithersburg, Md.) according to the manufacture's instructions. DNA strand breaks were quantitated by examining the fixed and stained cells under a fluorescence microscope (Olympus IX70) with 10X planoapo objectives. All analyses was performed with I.P.Lab Spectrum. The mean length of the DNA tail was determined by measuring 40 cells for each condition.

Poly(ADP-ribose) polymerase activity. Cells were incubated for 5 min at 37° C. in assay buffer containing 125 nmol NAD⁺ spiked with 0.25 μCi of ³H-NAD⁺ (Amersham) as described (12). Enzyme activity was determined by measuring cpm incorporated into protein.

Immunoprecipitation. BAECs were plated in 100 mm cell culture plates and grown to confluency. Cells (2×10⁷) were scraped from the plates, and 500 μg of protein was immunoprecipitated with 4 μg of the indicated antibody and 20 μl of Protein A Sepharose 4B (Amersham Phamacia Biotech) in binding buffer (final concentration 1 μg protein/ml).

Western blotting. Immunoprecipitated proteins electrophoresed on 10% PAGE gels were transferred onto nitrocellulose membranes. The immunoblots were developed with 1:1000 dilutions of anti-poly(ADPribose) IgG, and the signal was detected with the ECL System according to the manufacturer's instructions (Amersham Pharmacia Biotech). The images were scanned into a Molecular Dynamics FluorImager and analyzed using the ImageQuant 5.5 program.

Protein kinase C activity. The assay was performed according to the manufacturer's instructions using the Protein Kinase C Assay System (Invitrogen, Carlsbad, Calif.).

Hexosamine pathway activity. Hexosamine pathway activity was assessed by measuring UDP-GlcNAc concentration. Cells were homogenized in three volumes (600 μl) of cold 0.6 M perchloric acid and kept at 0° C. for 10 min. The precipitated proteins were removed by centrifugation for 5 min at 13,500×g, and UDP-GlcNAc in the supernatant was determined by HPLC as previously described (13).

Advanced glycation endproducts. Equal amounts of cell extract protein were used for quantitative immunoblotting performed as described (14). Methylglyoxal-derived imidazole advanced glycation endproducts were detected using monoclonal antibody 1H7G5 (identified above as IG7) at a 1:10,000 dilution. Immunocomplexes were visualized using an ECF kit according to the manufacturer's instructions (Amersham International, Piscataway, N.J.) and quantified using a Molecular Dynamics FluorImager and its ImageQuant 4.0 analytical software.

NFκB activation. A fluorescence in situ DNA-protein binding assay was performed in cultured cells as described (Kurose et al., 1997), and fluorescence per cell was determined using IP Lab Spectrum (Scanalytics, Fairfax, Va.).

Statistics. Data were analyzed using one-factor analysis of variance to compare the means of all the groups. The Tukey-Kramer multiple comparisons procedure was used to determine which pairs of means were different.

### Results

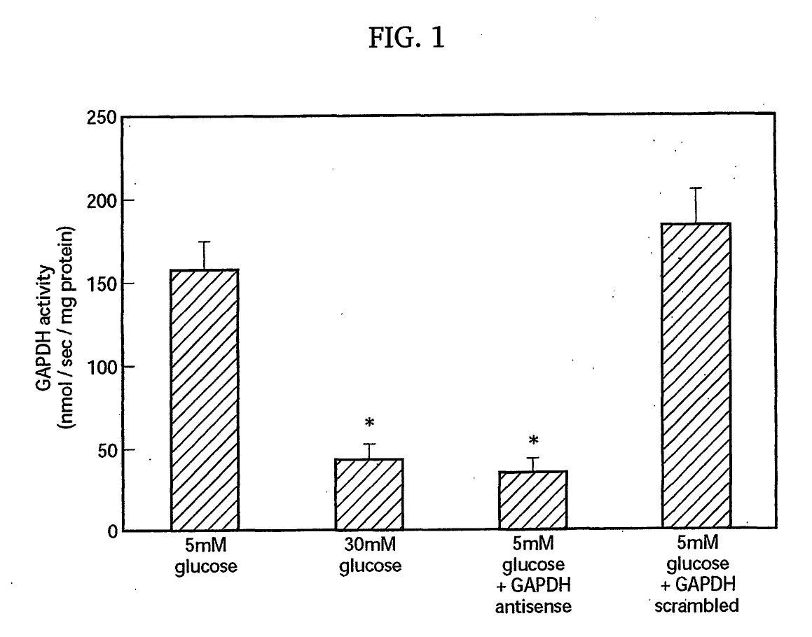

Specificity of GAPDH Antisense ODN. To test our hypothesis that hyperglycemia-induced mitochondrial superoxide overproduction activates pathways of hyperglycemic damage by partially inhibiting GAPDH and thereby diverting upstream glycolytic metabolites into these signaling pathways, we first needed to determine the specificity and efficacy of the GAPDH antisense oligonucleotides. As previously reported (Du et al., 2000), incubation of cells in 30 mM glucose reduced GAPDH activity by 73%, from 157.9±17.6 nmol/sec/mg protein (5 mM glucose) to 42.2±10.5 nmol/sec/mg protein (FIG. 1). After transfection with GAPDH antisense, cells incubated in 5 mM glucose showed a similar reduction in GAPDH activity, from 157.9±17.6 nmol/sec/mg protein (5 mM glucose) to 34.3±8.8 nmol/sec/mg protein (5 mM glucose+antisense). In contrast, after transfection with GAPDH scrambled oligonucleotides, GAPDH activity in cells incubated in 5 mM glucose was unchanged from that of cells incubated in 5 mM glucose alone (184.3±23.6 nmol/sec/mg protein (5 mM glucose+scrambled oligos) vs. 157.9±17.6 nmol/sec/mg protein (5 mM glucose alone).

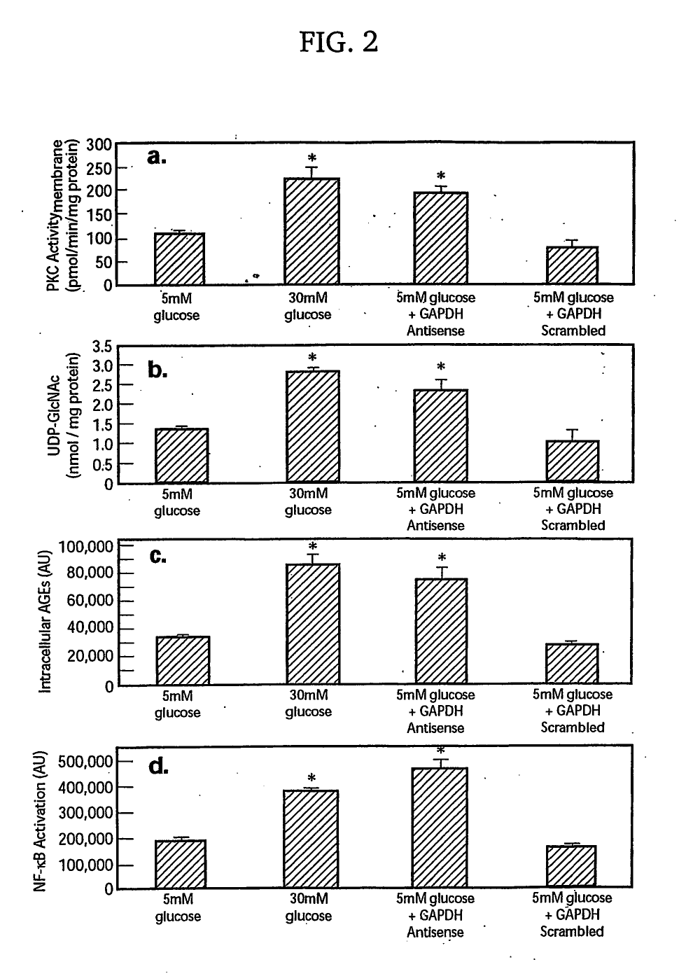

Effect of GAPDH Antisense ODN on Protein Kinase C Activation. Hexosamine Pathway Activation. Intracellular AGE formation, and NFκB Activation in Cells Cultured in 5 mM Glucose. Incubation of bovine aortic endothelial cells with 30 mM glucose increased the membrane fraction of intracellular PKC activity from 104.45±10.06 pmol/min/mg protein in cells incubated in 5 mM glucose (FIG. 2a, bar 1) to 224.54±23.94 pmol/min/mg protein (FIG. 2a, bar 2), as previously described (5). After transfection with GAPDH antisense (FIG. 2a, bar 3), cells incubated in 5 mM glucose showed a similar increase in PKC activity, from 104.45±10.06 pmol/min/mg protein (5 mM glucose) to 224.54±11.09 pmol/min/mg protein (5 mM glucose+antisense). In contrast, after transfection with GAPDH scrambled oligonucleotides (FIG. 2a, bar 4), PKC activity in cells incubated in 5 mM glucose was unchanged from that of cells incubated in 5 mM glucose alone (81.13±13.18 pmol/min/mg protein (5 mM glucose+scrambled ODN) vs. 104.45±10.06 pmol/min/mg protein (5 mM glucose alone).

Similarly, incubation of bovine aortic endothelial cells with 30 mM glucose increased the UDP-N-acetylglucosamine concentration, an indicator of hexosamine pathway flux, from 1.35±0.08 nmol/mg protein in cells incubated with 5 mM glucose (FIG. 2b, bar 1) to 2.82±0.12 nmol/mg protein (FIG. 2b, bar 2), as previously described (Du et al., 2000). After transfection with GAPDH antisense (FIG. 2b, bar 3), cells incubated in 5 mM glucose showed a similar increase in UDP-N-acetylglucosamine, from 1.35±0.08 nmol/mg protein (5 mM glucose) to 2.38±0.30 nmol/mg protein (5 mM glucose+antisense). In contrast, after transfection with GAPDH scrambled oligonucleotides (FIG. 2a, bar 4), UDP-N-acetylglucosamine concentration in cells incubated in 5 mM glucose+scrambled ODN was unchanged from that of cells incubated in 5 MM glucose alone (1.06±0.03 nmol/mg protein−5 mM glucose+scrambled ODN vs. 1.35±0.08 nmol/mg protein−5 mM glucose alone).

Incubation of bovine aortic endothelial cells with 30 mM glucose increased intracellular AGE formation from 33,315±1,750 arbitrary units in cells incubated in 5 mM glucose (FIG. 2c, bar 1) to 85,954±7,431 arbitrary units (FIG. 2c, bar 2), as previously described (5). This hyperglycemia-induced 2.6-fold increase was reproduced in cells transfected with GAPDH antisense (FIG. 2c, bar 3), (74,837±3,828 AU, FIG. 2c, bar 3), while in cells transfected with GAPDH scrambled ODN (FIG. 2c, bar 4), AGE formation was not different (27,238±3,819 AU) from that of cells incubated in 5 mM glucose alone.

Lastly, incubation of bovine aortic endothelial cells with 30 mM glucose increased NFκB activation by 2-fold, from 188,636±13,333 AU in cells incubated in 5 mM glucose (FIG. 2d, bar 1) to 379,053±9,734 AU in cells incubated in 30 mM glucose (FIG. 2d, bar 2), as previously described (Nishikawa et al., 2000). This hyperglycemia-induced increase was reproduced in cells transfected with GAPDH antisense (FIG. 2d, bar 3), (465,044±3,1474 AU), while in cells transfected with GAPDH scrambled ODN (FIG. 2d, bar 4), NFκB activation was not increased (160,009±13,388 AU) above that of cells incubated in 5 mM glucose alone.

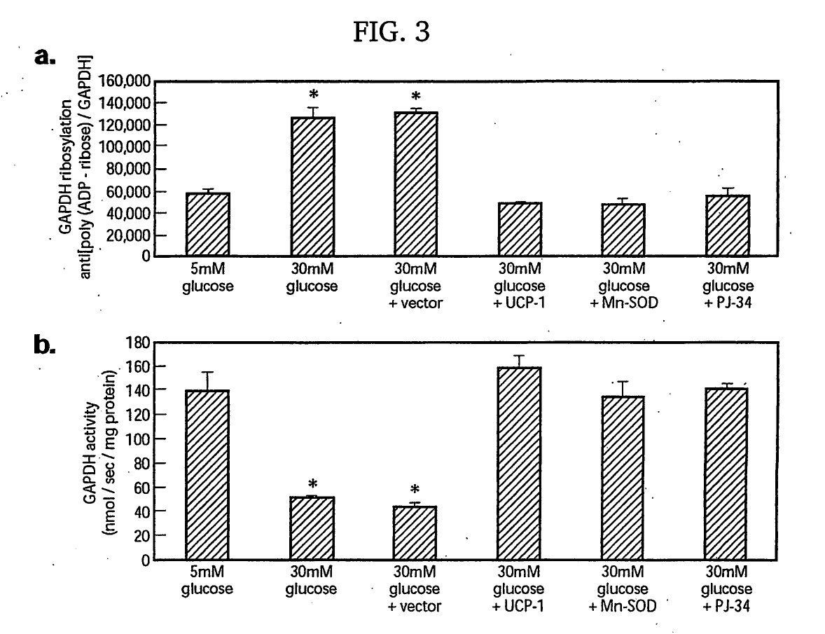

Effect of Poly(ADP-ribose) Polymerase Inhibition on GAPDH Activity. We next evaluated the extent of poly(ADP-ribosyl)ation on GAPDH in bovine aortic endothelial cells. Immunoprecipitation of GAPDH followed by western blotting with anti-polyADPribose IgG showed that incubation of cells in 30 mM glucose increased covalent modification of GAPDH by 2.2-fold, from 57,155±5,288 arbitrary units (FIG. 3A, bar 1) to 125,532±9,577 arbitrary units (FIG. 3A, bar 2). This increased modification of GAPDH by poly(ADP-ribosyl)ation was completely prevented in cells exposed to 30 mM glucose by overexpression of either uncoupling protein-1 (“UCP-1”)(FIG. 3A, bar 4), a specific protein uncoupler of oxidative phosphorylation capable of collapsing the proton electrochemical gradient which drives superoxide production (Nishikawa et al., 2000), or mangenese superoxide dismutase (Manna et al., 1998), the mitochondrial isoform of this enzyme (FIG. 3A, bar 5). The poly(ADP-ribosyl)ation of GADPH induced by 30 mM glucose was also completely prevented by addition of the potent poly(ADP-ribose) polymerase inhibitor, PJ34 (FIG. 3A, bar 6), the hydrochloride salt of N-(oxo-5,6-dihdrophenanthridin-2-yl)-N,N-dimethylacetamide (Pacher et al., 2002a). We then assessed the effect of this covalent modification on GAPDH activity (FIG. 3B). As described previously (Du et al., 2000), the inhibitory effect of incubation in 30 mM glucose was completely prevented by overexpression of either UCP-1 (FIG. 3B, bar 4) or MnSOD (FIG. 3B, bar 5), in parallel with the changes in poly(ADP-ribosyl)ation of GAPDH. The prevention of hyperglycemia-induced inhibition of GAPDH activity by PJ34 (FIG. 3B, bar 6) directly demonstrated that hyperglycemia-induced overproduction of superoxide inhibited GAPDH activity by causing poly(ADP-ribosyl)ation of the enzyme via poly(ADP-ribose) polymerase.

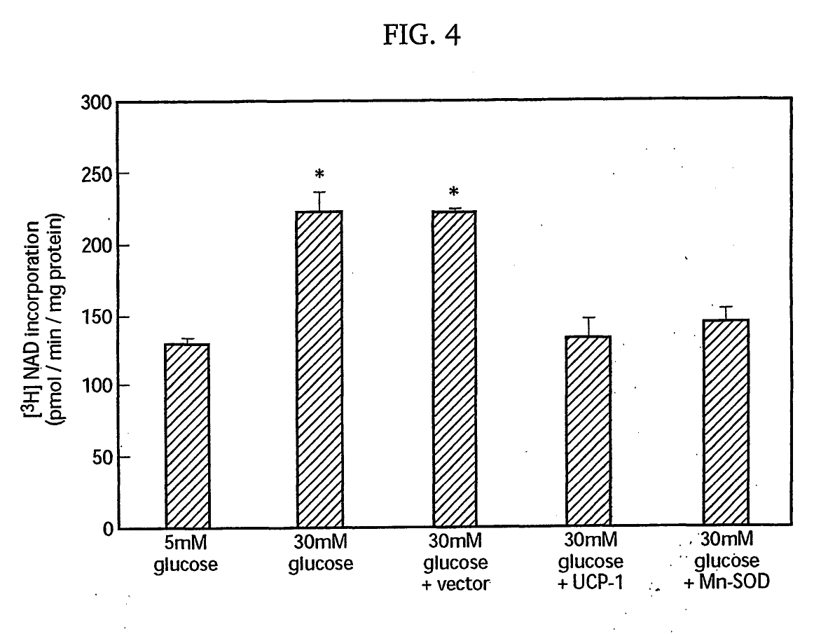

Effect of Hvperglycemia-Induced Mitochondrial Superoxide Overproduction on PARP Activity. To confirm directly that hyperglycemia-induced mitochondrial superoxide overproduction activated poly (ADP-ribose) polymerase, activity of this enzyme was determined (FIG. 4). Incubation of bovine aortic endothelial cells in 30 mM glucose increased PARP activity 1.7-fold, from 147.5±4.7 pmol/min/mg protein in cells incubated with 5 mM glucose (FIG. 4, bar 1) to 254.9±15.6 pmol/min/mg protein (FIG. 4, bar 2). The activation of PARP by 30 mM glucose was completely inhibited by overexpression of either UCP-1 (FIG. 4, bar 4), or MnSOD (FIG. 4, bar 5), to 152.1±15.1 pmol/min/mg protein and 165.5±9.5 pmol/min/mg protein, respectively, while vector alone had no effect.

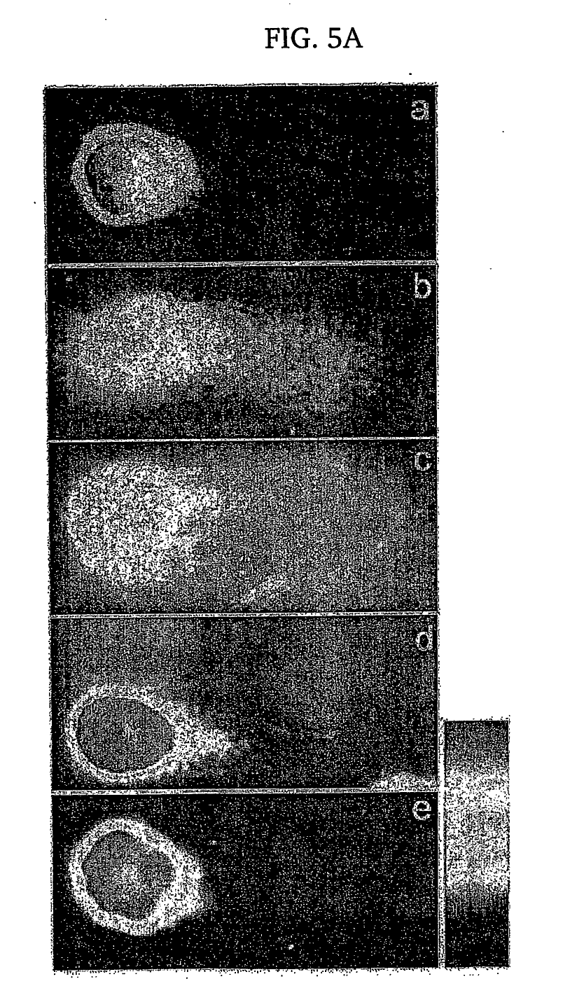

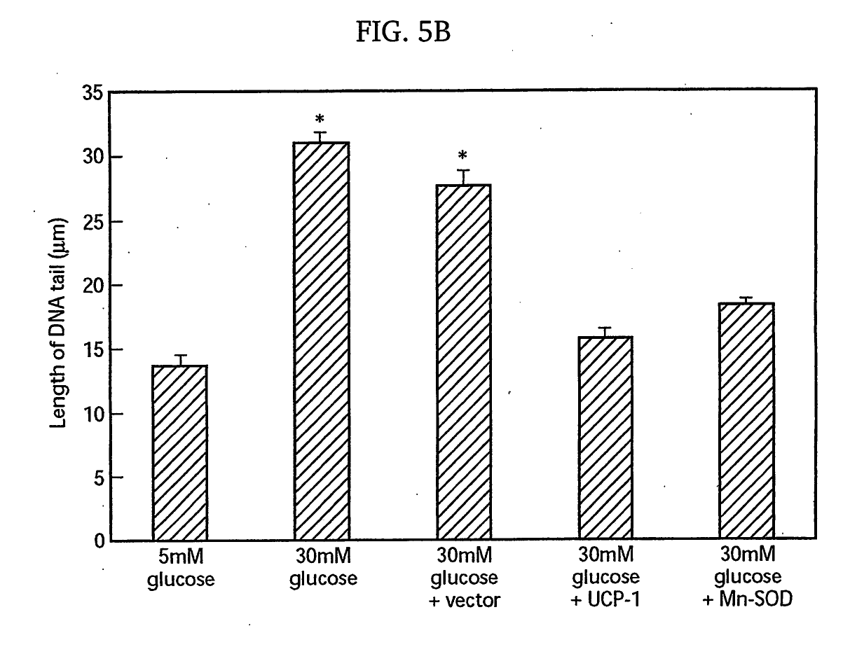

Effect of Hyperglycemia-Induced Mitochondrial Superoxide Overproduction on DNA Strand Breaks. Since PARP is activated by single or double strand breaks in DNA (Virag & Szabo, 2002), the effect of hyperglycemia-induced mitochondrial superoxide overproduction on DNA strand breaks was determined using the COMET single cell electrophoresis assay (FIG. 5A+B). Incubation in 30 mM glucose increased the length of the DNA tail two-fold, from 13.65±0.76 μm (FIG. 5A, panel 1, and 5B, bar 1) to 30.99±0.7 μm (FIG. 5A, panel 2, and 5B, bar 2). The increase in DNA strand breaks induced by 30 mM glucose was completely inhibited by overexpression of either UCP-1 (FIG. 5A, panel 4, and 5B, bar 4), or MnSOD (FIG. 5A, panel 5, and 5B, bar 5), to 15.67±0.77 μm and 18.27±0.57 μm, respectively, while vector alone had no effect.

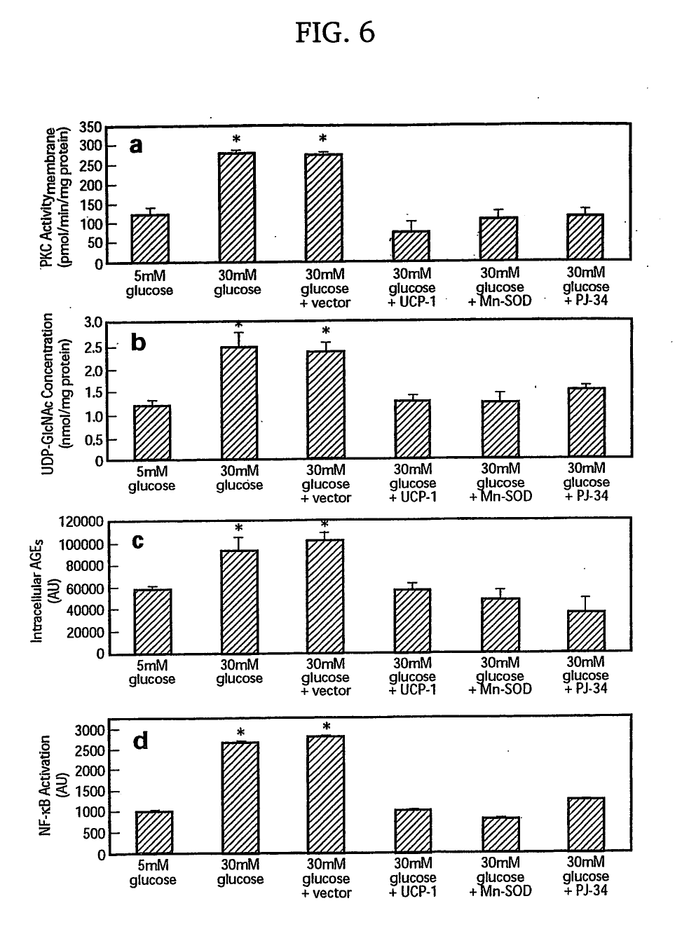

Effect of Poly (ADP-Ribose) Polymerase Inhibition on Protein Kinase C Activation. Hexosamine Pathway Activation, Intracellular AGE formation, and NFκB Activation in Cells Cultured in 30 mM Glucose. Having demonstrated that hyperglycemia-induced mitochondrial superoxide overproduction inhibits GAPDH activity by PARP-mediated poly(ADP-ribosyl)ation of the enzyme as a result of ROS-induced DNA strand breaks, and having shown that inhibiting GAPDH with antisense ODN activates multiple pathways of hyperglycemic damage in cells cultured in 5 mM glucose to the same extent as does culturing these cells in 30 mM glucose, we sought to determine whether this sequence of events explained the activation of these pathways by hyperglycemia in vivo (FIG. 6). We therefore evaluated the effect of PARP inhibition by PJ34 on each of the pathways of hyperglycemic damage in cells incubated with 30 mM glucose.

Incubation of bovine aortic endothelial cells in 30 mM glucose increased the membrane fraction of intracellular PKC activity from 123.79±17.3 pmol/min/mg protein in cells incubated in 5 mM glucose (FIG. 6a, bar 1) to 280.14±6.52 pmol/min/mg protein (FIG. 6a, bar 2). Overexpression of either UCP-1 or MNSOD completely inhibited the effect of 30 mM glucose, to 75.6±28.1 pmol/min/mg protein and 107.15±19.9 pmol/min/mg, respectively (FIG. 6a, bars 4 and 5), as shown previously (Nishikawa et al., 2000). Inhibition of PARP by PJ34 also completely prevented the activation of PKC by 30 mM glucose (FIG. 6a, bar 6). Similarly, incubation of bovine aortic endothelial cells with 30 mM glucose increased the UDP-N-acetylglucosamine concentration, an indicator of hexosamine pathway flux, from 1.18±0.13 nmol/mg protein in cells incubated with 5 mM glucose (FIG. 6b, bar 1) to 2.4±0.3 nmol/mg protein (FIG. 6b, bar 2). Overexpression of either UCP-1 or MnSOD completely inhibited the effect of 30 mM glucose, to 1.2±0.1 nmol/mg protein and 1.2±0.2 nmol/mg protein, respectively (FIG. 6b, bars 4 and 5), as shown previously (Du et al., 2000). Inhibition of PARP by PJ34 also completely prevented the effect of 30 mM glucose (FIG. 6b, bar 6). Incubation of bovine aortic endothelial cells with 30 mM glucose also increased intracellular AGE formation from 58107±3765 arbitrary units in cells incubated in 5 mM glucose (FIG. 6c, bar 1) to 92707±12906 arbitrary units (FIG. 6c, bar 2). Overexpression of either UCP-1 or MnSOD completely inhibited the effect of 30 mM glucose, to 56527±6319 arbitrary units and 48094±8739 arbitrary units, respectively (FIG. 6c, bars 4 and 5), as shown previously (Nishikawa et al., 2000). Inhibition of PARP by PJ34 also completely prevented the effect of 30 mM glucose (FIG. 6c, bar 6).

Lastly, incubation of bovine aortic endothelial cells with 30 mM glucose increased NFκB activation by 2.6-fold, from 1014±12 AU in cells incubated in 5 mM glucose (FIG. 6d, bar 1) to 2653±40 AU in cells incubated in 30 mM glucose (FIG. 6d, bar 2). Overexpression of either UCP-1 or MnSOD completely inhibited the effect of 30 mM glucose, to 997±4.9 arbitrary units and 798±3.8 arbitrary units, respectively (FIG. 6d, bars 4 and 5), as shown previously (Nishikawa et al., 2000). Inhibition of PARP by PJ34 also completely prevented this effect of 30 mM glucose (FIG. 6d, bar 6).

### Discussion

Four major hypotheses about how hyperglycemia causes diabetic complications have generated a large amount of data, as well as several clinical trials based on specific inhibitors of these mechanisms. The four hypotheses are activation of protein kinase C (PKC) isoforms, increased hexosamine pathway flux, increased advanced glycation endproduct (AGE) formation, and increased polyol pathway flux. Although specific inhibitors of individual pathways each ameliorate or prevent various diabetes-induced functional and structural abnormalities in cell culture and animal models, there has been no apparent common element linking the four mechanisms of hyperglycemia-induced damage (Hammes et al., 1991; Ishii et al., 1996; Lee et al., 1995; Nakamura et al., 1997; Soulis-Liparota et al., 1991). This issue has now been resolved by the recent discovery that each of the four different pathogenic mechanisms reflects a single hyperglycemia-induced process: overproduction of superoxide by the mitochondrial electron transport chain (Brownlee, 2001; Nishikawa et al., 2000).

In this report, we show that hyperglycemia-induced overproduction of superoxide by the mitochondrial electron transport chain activates the three major pathways of hyperglycemic damage found in aortic endothelial cells (activation of protein kinase C isoforms, hexosamine pathway flux, and advanced glycation endproduct [AGE] formation) by inhibiting GAPDH activity. Inhibition of GAPDH activity also activates the proinflammatory transcription factor NFκB, which in aortic endothelial cells is PKC dependent (Brownlee, 2001; Pieper & Riaz, 1997). This GAPDH inhibition is a consequence of poly(ADP-ribosyl)ation of GAPDH by poly(ADP-ribose) polymerase (PARP), which is activated by DNA strand breaks produced by mitochondrial superoxide overproduction. Hyperglycemia-induced activation of each of these pathways is completely inhibited by blocking PARP activity. Recent evidence has shown that glyceraldehyde-3-phosphate dehydrogenase (GAPDH) is not simply a classical glycolytic enzyme. Instead, it is a multifunctional protein with diverse cytoplasmic, membrane and nuclear activities (Sirover, 1997; Maxxola & Sirover, 2002). It is important to note that during cell death GAPDH translocates into the nucleus (Sawa et al., 1997; Schmitz, 2001), and recent work demonstrated that GAPDH can form complexes with nuclear proteins (Krynetski et al., 2003). Various modifications of the enzyme have been described including. its mono-ADP-ribosylation by nitric oxide (Brune & Lapetina, 1996). Here, using a combination of immunohistochemical and pharmacological tools, we demonstrate the poly(ADP-ribosyl)ation of the enzyme, which is likely to occur in association with its nuclear translocation.

Poly(ADP-ribose) polymerase is a nuclear DNA-repair enzyme that is activated by DNA strand breaks. Once activated, PARP catalyzes attachment of ADP ribose units from NAD⁺ to nuclear proteins, cleaving NAD⁺ into its component parts, ADP-ribose and nicotinamide mononucleotide. Replacement of this PARP-induced depletion of NAD⁺ consumes ATP. When PARP activation is excessive, this ATP consumption can cause cell death due to energy deficit (Eliasson et al., 1997). PARP-1 poly(ADP-ribosyl)ates transcription factors such as p53 and fos (Ha et al., 2002). However, PARP may also act as a coactivator or repressor of other transcription factors independent of its catalytic activity (Id.). Neither the enzymatic nor the DNA binding activity of PARP-1 is required for NFκB coactivator function (Id.; Hassa et al., 2002), for example, and PARP activity inhibitors fail to suppress inflammation-induced pro-IL-1β and ICAM-1 expression, while deletion of the PARP-1 gene does.

Thus, while mice lacking the PARP gene are resistant to injury from cerebral ischemia (Eliasson et al., 1997; Hassa et al., 2001) and myocardial ischemia-reperfusion (Pieper et al., 2000), it is not known whether these effects are due to altered PARP enzymatic activity or to altered protein-protein interactions with PARP. Hyperglycemia-induced NFκB activation does not occur in pulmonary microvascular endothelial cells of PARP-deficient mice, for example, while inhibition of PARP activity in wild-type pulmonary microvascular endothelial cells has no effect on hyperglycemia-induced NFκB activation (Garcia et al., 2001). In diabetic animal models, PARP inhibitors have been shown to improve myocardial dysfunction (Pacher et al., 2002b), and to prevent and reverse hyperglycemia-induced loss of endothelium-dependent vasodilatation (Id.; Soriano et al., 2001). Although PKC activation has been implicated in diabetic myocardial dysfunction (Wakasaki et al., 1997), and both PKC and the hexosamine pathway have been implicated in hyperglycemia-induced loss of endothelium-dependent vasodilatation (Beckman et al., 2002; Du et al. 2001), a possible link between PARP inhibition and activity of these pathways has not been investigated previously. Because there are two distinct mechanisms by which PARP affects cell function, poly(ADP-ribosyl)ation and protein-protein interaction, it has not been possible to predict whether PARP inhibition would have any effect on the development of long-term diabetic complications.

In animal models of long-term diabetic complications, it has been shown that treatment with a β isoform-specific PKC inhibitor ameliorates glomerular mesangial expansion (Koya et al., 2000), the pathognomonic structural feature of diabetic renal complications. It has also been shown that treatment of diabetic animal models with AGE inhibitors partially prevents various functional and structural manifestations of diabetic microvascular disease in retina, kidney, and nerve (Hammes et al., 1991; Nakamura et al., 1997; Soulis-Liparota et al., 1991) and that blockade of the AGE receptor in diabetic apoE-null mice significantly reduces atherosclerotic lesion size and structure (Bucciarelli et al., 2002; Park et al., 1998). The demonstration in this report that inhibition of PARP completely blocks hyperglycemia-induced activation of both of these pathways suggests that PARP inhibitors might have unique clinical efficacy in preventing the development and progression of diabetic complications.

### EXAMPLE 2

### Further Studies of the Mechanisms of the Pathology of Diabetic Complications

It is known that hyperglycemia is a major independent risk factor for accelerated atherosclerosis. The recent Insulin Resistance and Atherosclerosis Study (IRAS) demonstrates that there is also a strong link between insulin resistance itself and atherosclerosis, even in the absence of diabetes. Insulin resistance promotes atherosclerosis primarily by increasing free fatty acid release from, or defective uptake of free fatty acids into, adipocytes. This dysregulation of fatty acid metabolism causes a pro-atherogenic dyslipidemia by increasing free fatty acid delivery to the liver.

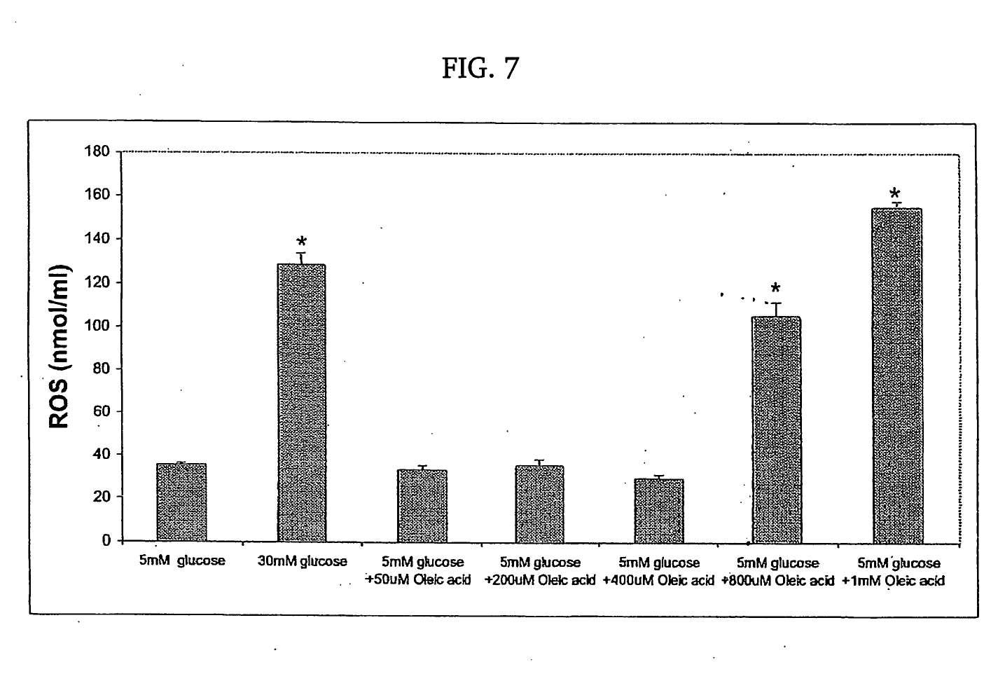

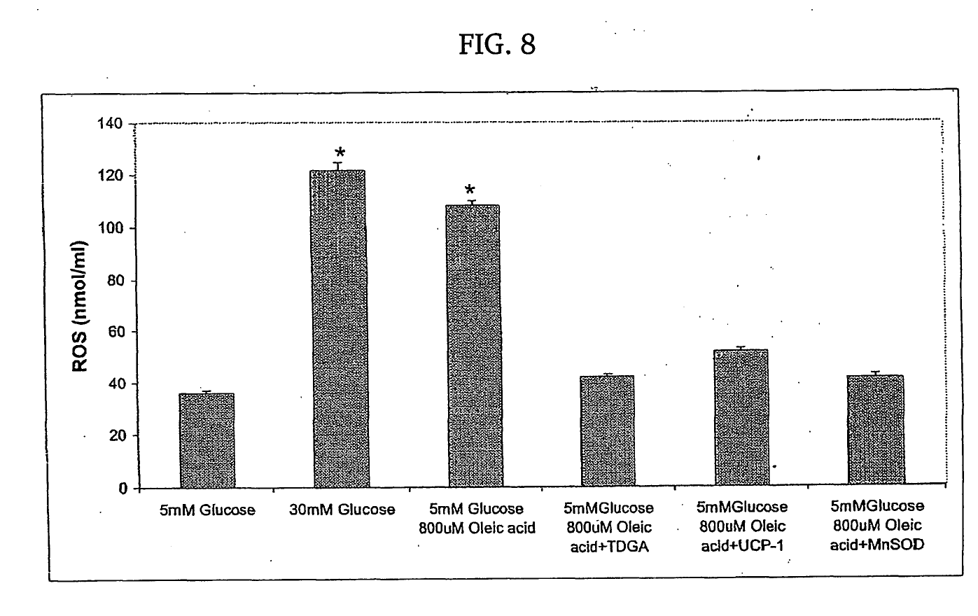

Recently, increased free fatty acids, like increased glucose, have been found to inhibit endothelial cell nitric oxide-dependent vasodilatation in normal subjects. Since mitochondrial β-oxidation of fatty acids yields the same electron donors as those obtained from glucose metabolism, that is, NADH and FADH₂, we hypothesized that increased free fatty acid flux from adipose tissue to vascular endothelium would also increase mitochondrial production of superoxide. In FIG. 7, we show that concentrations of free fatty acids (FFAs) found in insulin resistant and diabetic patients (bars 6 & 7) increase reactive oxygen species formation to the same extent as does hyperglycemia (bar 2), compared to normal glucose (bar 1) and normal FFA concentration (bars 3-5). In FIG. 8, we show that the ability of FFAs to induce reactive oxygen species in endothelial cells is inhibited by TDGA (bar 4), an inhibitor of carnitine palmitoyl transferase I (CPT-1), the rate limiting enzyme for transport of long-chain fatty acids into the mitochondria. The ability of FFAs to induce reactive oxygen species in endothelial cells is also blocked (bar 5) by overexpression of uncoupling protein-1 (UCP-1), which collapses the metabolite-generated proton electrochemical gradient across the mitochondrial inner membrane. This demonstrates that the mitochondrial electron transport chain is the source of hyperglycemia-induced reactive oxygen species (ROS) generation. Similarly, overexpression (bar 6) of manganese superoxide dismutase (MnSOD), the mitochondrial form of this enzyme, also abolished the reactive oxygen species signal; demonstrating that superoxide is the radical produced by hyperglycemia by the electron transport chain.

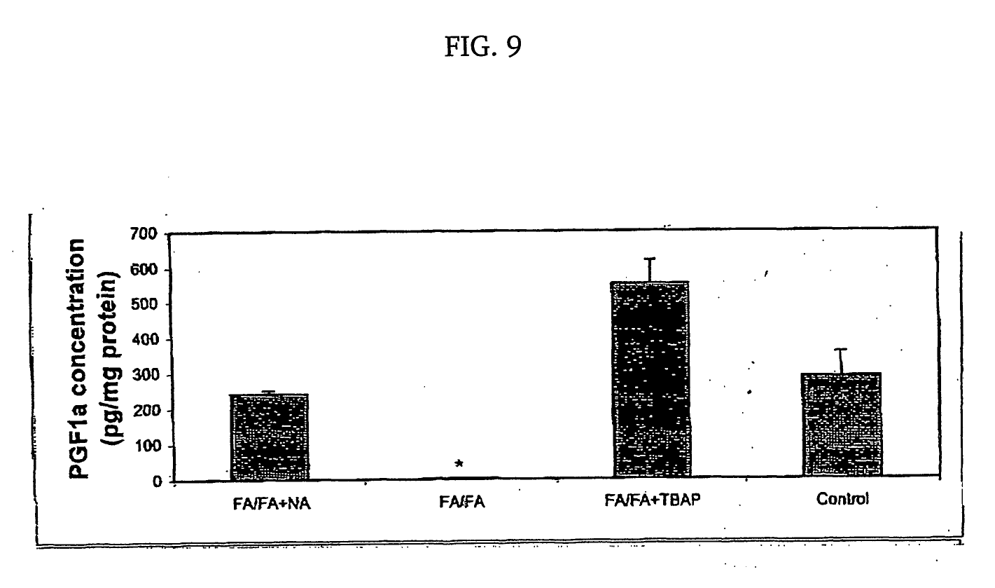

In order to examine whether this phenomenon was present in vivo, and thus had direct clinical relevance, we studied an accepted model of non-diabetic insulin resistance, the Fatty Zucker Rat. In these rats, we examined the activity of the endothelial cell surface enzyme prostacyclin synthetase, which produces a major anti-atherosclerotic product, prostacyclin. This enzyme is known to be directly inactivated by reactive oxygen species. Fatty Zucker Rats had only 1% of the activity in their aortas (FIG. 9, bar 2) that control Zucker rats had (FIG. 9, bar 4). This demonstrates that the insulin resistance rats have overproduction of reactive oxygen species in their endothelial cells that had inactivated the enzyme. When these rats were treated with the small molecular weight superoxide dismutase mimetic compound MnTBAP for one week, the activity of prostacyclin synthetase in aortas of Fatty Zucker Rats was restored to supranormal levels (FIG. 9, bar 3). This demonstrates that the enzyme was inactivated by overproduction of superoxide. Similarly, when the rats were treated for one week with the anti-lipolytic compound nicotinic acid (FIG. 9, bar 1), the activity of prostacyclin synthetase activity was restored to normal levels. This demonstrates that it is the increased release of FFAs due to insulin resistance that is responsible for the overproduction of superoxide in the aortas where the increased flux of FFAs is oxidized in the mitochondria.

It is well recognized clinically that diabetic patients with the same degree of hyperglycemia differ dramatically in their susceptibility to hyperglycemic damage. While the genetic determinants of susceptibility to both microvascular and macrovascular complications are not yet known, a role for genetic determinants of susceptibility to microvascular complications has been established by the demonstration of familial clustering of diabetic nephropathy and retinopathy. In two studies of families with two or more siblings having type 1 diabetes, if one diabetic sibling had advanced diabetic nephropathy, the other diabetic sibling had a nephropathy risk of 83% or 72%, while the risk was only 17% or 22% if the index case did not have diabetic nephropathy. For retinopathy, the DCCT reported familial clustering with an odds ratio of 5.4 for the risk of severe retinopathy in diabetic relatives of positive versus negative subjects from the conventional treatment group. For macrovascular complications, coronary artery calcification, an indicator of subclinical atherosclerosis, also shows familial clustering, with an estimated heritability of at least 40%.

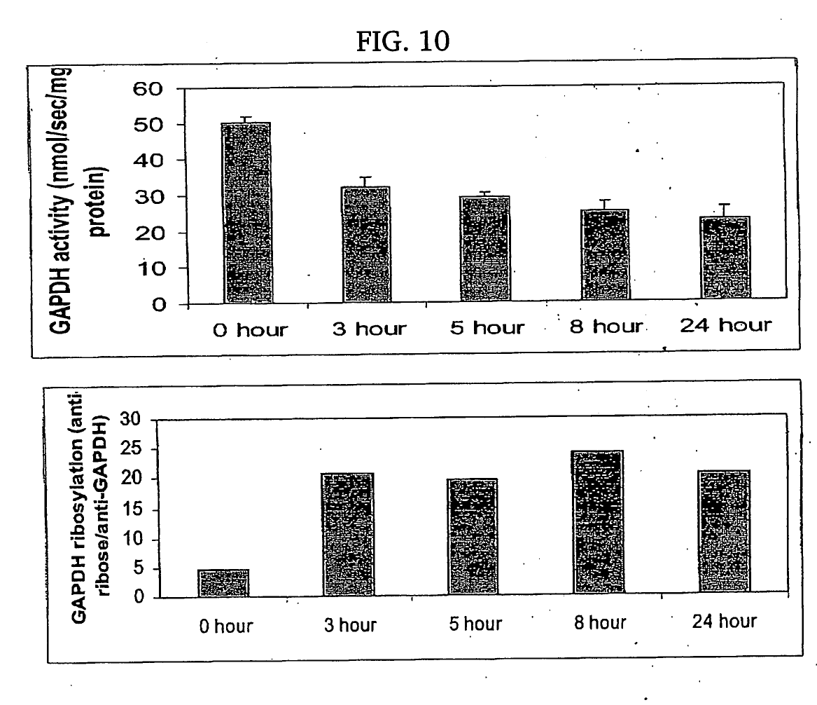

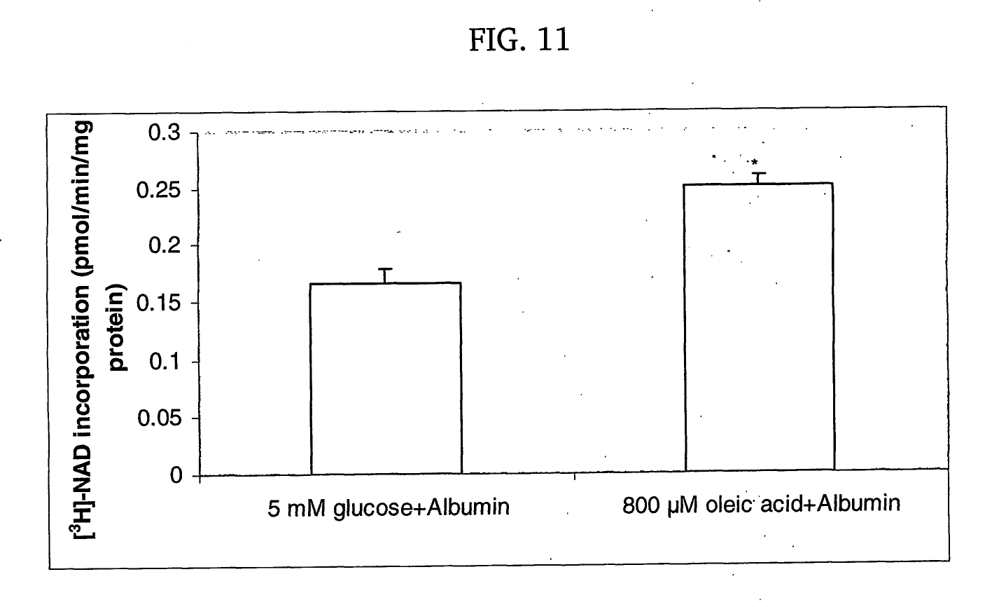

In order to determine whether there is a novel non-genetic marker for susceptibility, we performed studies in normal lean human subjects in their 20's with normal glucose metabolism. These subjects were infused with either diabetic levels of glucose, or insulin-resistant levels of FFAs, for 4-6 hours. At the beginning of these studies, and at various times during and after the study, we measured activity of the enzyme glyceraldehyde-3-phosphate dehydrogenase (GAPDH, the enzyme whose decrease in activity due to ADP-ribosylation of the enzyme by ROS-activated poly(ADP-ribose) polymerase (PARP). We also measured the extent of GAPDH poly(ADP-ribosyl)ation. As shown in FIG. 10, upper panel, the activity of GAPDH decreases significantly by 3 hours after the start of the metabolite infusion, and in some subjects, does not normalize even after the infusion is terminated. This effect can last for at least 24 hours. Concomitant with the loss of GAPDH activity, there is a reciprocal increase in GAPDH ADP-ribosylation (FIG. 10, lower panel). These data demonstrate that not only hyperglycemia activates PARP, as we show in endothelial cells in FIG. 4, but that free fatty acids at levels found in non-diabetic insulin resistance subjects also activate PARP to the same extent as hyperglycemia. As shown in FIG. 11, incubation of aortic endothelial cells with oleic acid at these levels increases PARP activation by 1.5-fold.

Thus, the activity, poly(ADP-ribosyl)ation, and its consequences, activation of protein kinase C, the hexosamine pathway (N-acetylglucosamine protein modification), and advanced glycation endproduct modification of proteins, can all be used as markers of individual susceptibility to their ambient level of hyperglycemia and/or free fatty acids, and of the differential response of that individual to various therapeutic modalities for either the primary treatment of diabetes, or for the prevention of vascular dysfunction and disease.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

SEQ ID NO:1 - mRNA of human PARP GenBank NM 001618.

| | |
|---|---|
| 1 | aatctatcag ggaacggcgg tggccggtgc ggcgtgttcg gtgcgctctg gccgctcagg |
| 61 | ccgtgcggct gggtgagcgc acgcgaggcg gcgaggcggc aagcgtgttt ctaggtcgtg |
| 121 | gcgtcgggct tccggagctt tggcggcagc taggggagga tggcggagtc ttcggataag |
| 181 | ctctatcgag tcgagtacgc caagagcggg cgcgcctctt gcaagaaatg cagcgagagc |
| 241 | atccccaagg actcgctccg gatggccatc atggtgcagt cgcccatgtt tgatggaaaa |
| 301 | gtcccacact ggtaccactt ctcctgcttc tggaaggtgg gccactccat ccggcaccct |
| 361 | gacgttgagg tggatgggtt ctctgagctt cggtgggatg accagcagaa agtcaagaag |
| 421 | acagcggaag ctggaggagt gacaggcaaa ggccaggatg gaattggtag caaggcagag |
| 481 | aagactctgg gtgactttgc agcagagtat gccaagtcca acagaagtac gtgcaagggg |
| 541 | tgtatggaga agatagaaaa gggccaggtg cgcctgtcca agaagatggt ggacccggag |
| 601 | aagccacagc taggcatgat tgaccgctgg taccatccag gctgctttgt caagaacagg |
| 661 | gaggagctgg gtttccggcc cgagtacagt gcgagtcagc tcaagggctt cagcctcctt |
| 721 | gctacagagg ataaagaagc cctgaagaag cagctcccag gagtcaagag tgaaggaaag |
| 781 | agaaaaggcg atgaggtgga tggagtggat gaagtggcga agaagaaatc taaaaaagaa |
| 841 | aaagacaagg atagtaagct tgaaaaagcc ctaaaggctc agaacgacct gatctggaac |
| 901 | atcaaggacg agctaaagaa agtgtgttca actaatgacc tgaaggagct actcatcttc |
| 961 | aacaagcagc aagtgccttc tggggagtcg gcgatcttgg accgagtagc tgatggcatg |
| 1021 | gtgttcggtg ccctccttcc ctgcgaggaa tgctcgggtc agctggtctt caagagcgat |
| 1081 | gcctattact gcactgggga cgtcactgcc tggaccaagt gtatggtcaa gacacagaca |
| 1141 | cccaaccgga aggagtgggt aaccccaaag gaattccgag aaatctctta cctcaagaaa |
| 1201 | ttgaaggtta aaaagcagga ccgtatattc cccccagaaa ccagcgcctc cgtggcggcc |
| 1261 | acgcctccgc cctccacagc ctcggctcct gctgctgtga actcctctgc ttcagcagat |
| 1321 | aagccattat ccaacatgaa gatcctgact ctcgggaagc tgtcccggaa caaggatgaa |
| 1381 | gtgaaggcca tgattgagaa actcgggggg aagttgacgg ggacggccaa caaggcttcc |
| 1441 | ctgtgcatca gcaccaaaaa ggaggtggaa aagatgaata agaagatgga ggaagtaaag |
| 1501 | gaagccaaca tccgagttgt gtctgaggac ttcctccagg acgtctccgc ctccaccaag |
| 1561 | agccttcagg agttgttctt agcgcacatc ttgtcccctt ggggggcaga ggtgaaggca |
| 1621 | gagcctgttg aagttgtggc cccaagaggg aagtcagggg ctgcgctctc caaaaaaagc |
| 1681 | aagggccagg tcaaggagga aggtatcaac aaatctgaaa agagaatgaa attaactctt |
| 1741 | aaaggaggag cagctgtgga tcctgattct ggactggaac actctgcgca tgtcctggag |
| 1801 | aaaggtggga aggtcttcag tgccaccctt ggcctggtgg acatcgttaa aggaaccaac |
| 1861 | tcctactaca agctgcagct tctggaggac gacaaggaaa acaggtattg gatattcagg |
| 1921 | tcctggggcc gtgtgggtac ggtgatcggt agcaacaaac tggaacagat gccgtccaag |
| 1981 | gaggatgcca ttgagcagtt catgaaatta tatgaagaaa aaaccgggaa cgcttggcac |
| 2041 | tccaaaaatt tcacgaagta tcccaaaaag ttttaccccc tggagattga ctatggccag |
| 2101 | gatgaagagg cagtgaagaa gctcacagta aatcctggca ccaagtccaa gctccccaag |
| 2161 | ccagttcagg acctcatcaa gatgatcttt gatgtggaaa gtatgaagaa agccatggtg |
| 2221 | gagtatgaga tcgaccttca gaagatgccc ttggggaagc tgagcaaaag gcagatccag |
| 2281 | gccgcatact ccatcctcag tgaggtccag caggcggtgt ctcagggcag cagcgactct |
| 2341 | cagatcctgg atctctcaaa tcgcttttac accctgatcc cccacgactt tgggatgaag |
| 2401 | aagcctccgc tcctgaacaa tgcagacagt gtgcaggcca aggtggaaat gcttgacaac |
| 2461 | ctgctggaca tcgaggtggc ctacagtctg ctcaggggag ggtctgatga tagcagcaag |
| 2521 | gatcccatcg atgtcaacta tgagaagctc aaaactgaca ttaaggtggt tgacagagat |
| 2581 | tctgaagaag ccgagatcat caggaagtat gttaagaaca ctcatgcaac cacacacagt |
| 2641 | gcgtatgact tggaagtcat cgatatcttt aagatagagc gtgaaggcga atgccagcgt |
| 2701 | tacaagccct ttaagcagct tcataaccga agattgctgt ggcacgggtc caggaccacc |
| 2761 | aactttgctg ggatcctgtc ccagggtctt cggatagccc cgcctgaagc gcccgtgaca |
| 2821 | ggctacatgt ttggtaaagg gatctatttc gctgacatgg tctccaagag tgccaactac |
| 2881 | taccatacgt ctcagggaga cccaataggc ttaatcctgt tgggagaagt tgcccttgga |
| 2941 | aacatgtatg aactgaagca cgcttcacat atcagcaggt tacccaaggg caagcacagt |
| 3001 | gtcaaaggtt tgggcaaaac tacccctgat ccttcagcta acattagtct ggatggtgta |
| 3061 | gacgttcctc ttgggaccgg gatttcatct ggtgtgatag acacctctct actatataac |
| 3121 | gagtacattg tctatgatat tgctcaggta aatctgaagt atctgctgaa actgaaattc |
| 3181 | aattttaaga cctccctgtg gtaattggga gaggtagccg agtcacaccc ggtggctgtg |
| 3241 | gtatgaattc acccgaagcg cttctgcacc aactcacctg gccgctaagt tgctgatggg |
| 3301 | tagtacctgt actaaaccac ctcagaaagg attttacaga aacgtgttaa aggttttctc |
| 3361 | taacttctca agtcccttgt tttgtgttgt gtctgtgggg aggggttgtt ttggggttgt |
| 3421 | ttttgttttt tcttgccagg tagataaaac tgacatagag aaaaggctgg agagagattc |
| 3481 | tgttgcatag actagtccta tggaaaaaac caaagcttcg ttagaatgtc tgccttactg |
| 3541 | gtttccccag ggaaggaaaa atacacttcc accctttttt ctaagtgttc gtctttagtt |
| 3601 | ttgattttgg aaagatgtta agcatttatt tttagttaaa ataaaaacta atttcatact |
| 3661 | atttagattt tcttttttat cttgcactta ttgtcccctt tttagttttt tttgtttgcc |
| 3721 | tcttgtggtg aggggtgtgg gaagaccaaa ggaaggaacg ctaacaattt ctcatactta |
| 3781 | gaaacaaaaa gagctttcct tctccaggaa tactgaacat gggagctctt gaaatatgta |
| 3841 | gtattaaaag ttgcatttg |

1. A method of inhibiting the development or progression of atherosclerotic, microvascular, or neurologic disease due to diabetes or insulin resistance in a mammal, or conditions resulting therefrom, the method comprising specifically inhibiting poly(ADP-ribose) polymerase (PARP) activity or accumulation in the mammal for a time sufficient to inhibit the development or progression of the disease or condition.

2. The method of claim 1, wherein the disease or condition is selected from the group consisting of coronary disease, myocardial infarction, atherosclerotic peripheral vascular disease, cerebrovascular disease, stroke, retinopathy, renal disease, neuropathy, and cardiomyopathy.

3. The method of claim 1, wherein the condition is retinopathy.

4. The method of claim 1, wherein the PARP activity is inhibited by administering to the mammal a PARP inhibitor.

5. The method of claim 4, wherein the PARP inhibitor is selected from the group consisting of PJ34, 3-aminobenzamide, 4-amino-1,8-naphthalimide, 6(5H)-phenanthridinone, benzamide, INO-1001, and NU1025.

6. (canceled)

7. The method of claim 1, wherein the PARP activity is inhibited by administering to the mammal a nucleic acid or mimetic that specifically inhibits transcription or translation of the PARP gene.

8. The method of claim 7, wherein the nucleic acid or mimetic is selected from the group consisting of an antisense complementary to mRNA of the PARP gene, a ribozyme capable of specifically cleaving the mRNA of the PARP gene, and an RNAi molecule complementary to a portion of the PARP gene, wherein the PARP gene is at least 80% homologous to SEQ ID NO:1.

9. The method of claim 1, wherein the PARP activity is inhibited by administration of a compound that specifically binds to the PARP.

10. (canceled)

11. The method of claim 1, further comprising activating transketolase in the mammal.

12. The method of claim 11, wherein transketolase is activated by administering a lipid-soluble thiamine derivative to the mammal.

13. The method of claim 12, wherein the lipid-soluble thiamine derivative is selected from the group consisting of benfotiamine, thiamine propyl disulfide, and thiamine tetrahydrofurfuryl disulfide.

14. The method of claim 1, further comprising reducing superoxide or peroxynitrite in the mammal.

15. The method of claim 14, wherein the superoxide or peroxynitrite is reduced in the mammal by administering to the mammal a compound selected from the group consisting of R-alpha-lipoic acid, FP15, a superoxide dismutase mimetic and a catalase mimetic.

16. The method of claim 15, wherein the compound is a superoxide dismutase mimetic or a catalase mimetic selected from the group consisting of MnTBAP, ZnTBAP, SC-55858, EUK-134, M40403, AEOL 10112, AEOL 10113, and AEOL 10150.

17. (canceled)

18. The method of claim 1, further comprising inhibiting excessive release of free fatty acids in the mammal.

19. The method of claim 18, wherein excessive release of free fatty acids is inhibited by administering to the mammal a compound selected from the group consisting of a thiazolidinedione, nicotinic acid, adiponectin and acipimox.

20. An antibody preparation comprising antibodies that specifically react with Nα-acetyl-Nδ(5-hydro-5-methyl)-4-imidazolone.

21-22. (canceled)

23. The antibody preparation of claim 20, wherein the antibodies are IG7 monoclonal antibodies.

24. A method of monitoring the effectiveness of an anti-diabetic or anti-insulin resistance treatment or an anti-diabetic or anti-insulin resistance complication treatment in a mammal, the method comprising measuring ADP-ribosylated protein levels and/or GlcNAc-modified protein levels in the mammal before and after the treatment, wherein ADP-ribosylated protein levels and/or GlcNAc-modified protein levels after the treatment lower than ADP-ribosylated protein levels and/or GlcNAc-modified protein levels before the treatment indicates that the treatment is effective.

25-36. (canceled)

37. A method of monitoring the effectiveness of an anti-diabetic or anti-insulin resistance treatment or anti-diabetic or anti-insulin resistance complication treatment in a mammal, the method comprising measuring methylglyoxyl AGE levels in the mammal using the antibody preparation of claim 20, wherein methylglyoxyl AGE levels after the treatment lower than methylglyoxyl AGE levels before the treatment indicates that the treatment is effective.

38-61. (canceled)


SEQUENCE LISTING (3 sequences)

SEQ ID NO: 1 - DNA, 3859 bp, Homo sapiens
aatctatcagggaacggcggtggccggtgcggcgtgttcggtgcgctctggccgctcaggccgtgcggctgggtgagcgcacgcgaggcggcgaggcggcaagcgtgtttctaggtcgtggcgtcgggcttccggagctttggcggcagctaggggaggatggcggagtcttcggataagctctatcgagtcgagtacgccaagagcgggcgcgcctcttgcaagaaatgcagcgagagcatccccaaggactcgctccggatggccatcatggtgcagtcgcccatgtttgatggaaaagtcccacactggtaccacttctcctgcttctggaaggtgggccactccatccggcaccctgacgttgaggtggatgggttctctgagcttcggtgggatgaccagcagaaagtcaagaagacagcggaagctggaggagtgacaggcaaaggccaggatggaattggtagcaaggcagagaagactctgggtgactttgcagcagagtatgccaagtccaacagaagtacgtgcaaggggtgtatggagaagatagaaaagggccaggtgcgcctgtccaagaagatggtggacccggagaagccacagctaggcatgattgaccgctggtaccatccaggctgctttgtcaagaacagggaggagctgggtttccggcccgagtacagtgcgagtcagctcaagggcttcagcctccttgctacagaggataaagaagccctgaagaagcagctcccaggagtcaagagtgaaggaaagagaaaaggcgatgaggtggatggagtggatgaagtggcgaagaagaaatctaaaaaagaaaaagacaaggatagtaagcttgaaaaagccctaaaggctcagaacgacctgatctggaacatcaaggacgagctaaagaaagtgtgttcaactaatgacctgaaggagctactcatcttcaacaagcagcaagtgccttctggggagtcggcgatcttggaccgagtagctgatggcatggtgttcggtgccctccttccctgcgaggaatgctcgggtcagctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaagcaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcagttcatgaaattatatgaagaaaaaaccgggaacgcttggcactccaaaaatttcacgaagtatcccaaaaagttttaccccctggagattgactatggccaggatgaagaggcagtgaagaagctcacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacagtgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactaccatacgtctcagggagacccaataggcttaatcctgttgggagaagttgcccttggaaacatgtatgaactgaagcacgcttcacatatcagcaggttacccaagggcaagcacagtgtcaaaggtttgggcaaaactacccctgatccttcagctaacattagtctggatggtgtagacgttcctcttgggaccgggatttcatctggtgtgatagacacctctctactatataacgagtacattgtctatgatattgctcaggtaaatctgaagtatctgctgaaactgaaattcaattttaagacctccctgtggtaattgggagaggtagccgagtcacacccggtggctgtggtatgaattcacccgaagcgcttctgcaccaactcacctggccgctaagttgctgatgggtagtacctgtactaaaccacctcagaaaggattttacagaaacgtgttaaaggttttctctaacttctcaagtcccttgttttgtgttgtgtctgtggggaggggttgttttggggttgtttttgttttttcttgccaggtagataaaactgacatagagaaaaggctggagagagattctgttgcatagactagtcctatggaaaaaaccaaagcttcgttagaatgtctgccttactggtttccccagggaaggaaaaatacacttccacccttttttctaagtgttcgtctttagttttgattttggaaagatgttaagcatttatttttagttaaaataaaaactaatttcatactatttagattttcttttttatcttgcacttattgtcccctttttagttttttttgtttgcctcttgtggtgaggggtgtgggaagaccaaaggaaggaacgctaacaatttctcatacttagaaacaaaaagagctttccttctccaggaatactgaacatgggagctcttgaaatatgtagtattaaaagttgcatttg

SEQ ID NO: 2 - DNA, 20 bp, Artificial
  synthesized
gtagaagcagggatgatatt

SEQ ID NO: 3 - DNA, 20 bp, Artificial
  synthesized
gaataagtgatacggatgtg